(19) United States
(12) Patent Application Publication
(10) Pub. No.: US 2025/0204939 A1
(43) Pub. Date: Jun. 26, 2025

(54) VISUALIZING CLOT COLLECTION IN A THROMBECTOMY SYSTEM

(71) Applicant: Walk Vascular, LLC, Irvine, CA (US)

(72) Inventors: Gioconda Alvarado, San Jose, CA (US); Seung Hae Clare Chung, Los Altos, CA (US); Rafael A. Castellanos Alvarez, San Jose, CA (US); Nora V. Legarda, San Jose, CA (US); Robert Kai Rounds, Sunnyvale, CA (US)

(21) Appl. No.: 18/978,264

(22) Filed: Dec. 12, 2024

Related U.S. Application Data
(60) Provisional application No. 63/613,911, filed on Dec. 22, 2023.

(51) Int. Cl. A61B 17/22; A61M 1/00

(52) U.S. Cl. CPC A61B 17/22; A61M 1/71; A61B 2017/22079

(57) ABSTRACT

Disclosed are embodiments of a vacuum canister configured to provide negative pressure to an aspiration catheter. The vacuum canister is also configured to received aspirant from the aspiration catheter and to separate the aspirant constituents (including thrombus, blood, and saline) for visual confirmation, returning the patient's own blood, and later analysis of collected thrombus.

### CROSS-REFERENCE TO RELATED APPLICATIONS

This application claims priority to and the benefit of U.S. Provisional Patent Application Ser. No. 63/613,911, filed Dec. 22, 2023, and entitled “VISUALIZING CLOT COLLECTION IN A THROMBECTOMY SYSTEM,” the disclosure of which is incorporated herein by this reference.

### BACKGROUND OF THE INVENTION

### Technical Field

The present disclosure pertains generally to medical devices and methods of their use. More particularly, the present invention pertains to aspiration and thrombectomy devices and methods of use thereof.

### Description of the Related Art

Several devices and systems already exist to aid in the removal of thrombotic material. These include simple aspiration-tube-type devices using vacuum syringes to extract thrombus into the syringe, simple flush-and-aspirate devices, and more complex devices with rotating components that pull in, macerate and transport thrombotic material away from the distal tip using a mechanical auger.

All of the devices described above have limitations as a result of individual design characteristics. For example, simple aspiration catheters offer ease of use and rapid deployment but may become blocked or otherwise inoperable when faced with older, more organized thrombotic material. Such devices must be removed and cleared outside the body and then re-inserted into the vasculature, which lengthens the time needed for the procedure and increases the opportunity to kink the catheter shaft. Such kinks may reduce performance by decreasing the cross-sectional area of the catheter or may render the device inoperable.

Mechanical rotary devices use an auger to grab and carry the thrombus away from the target area. Some create transport force via vacuum bottles while others create differential pressure at the distal tip of the device with the auger acting as a low-pressure pump. These devices typically work slowly and offer the physician no feedback as to when the device should be advanced further into the lesion.

Flushing type devices include manual flush type devices in which the physician manipulates a hand-driven pump to provide flowing saline at the tip of the device to break up and aspirate the thrombus material, which may introduce performance variations based on the ability of the physician to consistently pump the device over the duration of the procedure.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Implementations of the present invention solve one or more problems in the art with systems, methods, and apparatus for aspirating thrombus. Disclosed are embodiments of vacuum canisters configured for use in a thrombectomy system, the canister comprising a receptacle configured to receive aspirant from a thrombectomy system, a lid configured to attach to the receptacle, the lid having one or more ports, one or more separation members configured to attach to an interior wall of the receptacle, and wherein the separation member is configured to separate thrombus from aspirant. Separation of thrombus from aspirant beneficially facilitates visualization of the thrombus and/or collection of the thrombus for analysis. The vacuum canister may be placed under vacuum and be configured to communicate a vacuum to the aspiration lumen of an aspiration catheter. The separation member may comprise a plurality of apertures configured to allow the passage of blood and saline of aspirant, but not thrombus, to pass therethrough. In some embodiments, the separation member may comprise a mesh or permeable membrane. The aspiration member may be configured to conform to a wall of the receptacle such that all aspirant must pass through the separation member before reaching the bottom of the receptacle. The separation member may have a substantially flat or curved surface for directing thrombus to a location upon a surface of the separation member. The separation member may be disposed within the canister at an oblique angle relative to a bottom surface of the lid of the canister to promote movement of aspirant over the surface of the separation member.

In some embodiments, the vacuum canister may comprise a receptacle configured to receive aspirant from a thrombectomy system, a lid configured to attach to the receptacle to form an interior space, the lid having one or more ports, a moveable separation member, at least a portion of which is disposed within the interior space, and wherein the moveable separation member is configured to deliver aspirant received into the vacuum canister onto an interior wall of the receptacle. The moveable separation member may comprise a rotating filter, tray, impeller, fan or ladle. The moveable separation member may be connected to a motor, such that the moveable separation member may rotate within the canister. The moveable separation member may be disposed beneath a port connected to the aspiration lumen, such that aspirant passing through the port is dispersed by the moveable separation member upon entering the canister. The aspirant may be dispersed over the walls of the canister or over the surface of a separation member. In some embodiments, the moveable separation member may comprise vertical and slanted walls configured for collecting thrombus. In some embodiments, the moveable separation member may comprise a plurality of apertures configured to separate thrombus from aspirant.

In some embodiments, the vacuum canister may comprise a receptacle configured to receive aspirant from a thrombectomy system, a lid configured to attach to the receptacle to form an interior space and wherein the lid includes a port, an alternate separation member, at least a portion of which is disposed within the interior space, wherein the alternate separation member is configured to connect to the lid, wherein the alternate separation member is disposed over an outlet of the port, and wherein the alternate separation member is configured to separate thrombus from aspirant received through the port.

The alternate separation member may comprise a collection sack having one or more permeable walls. The permeable walls may include a plurality of apertures configured to separate thrombus from aspirant. In some embodiments, the alternate separation member may comprise multiple permeable walls. In some embodiments, the alternate separation member may comprise a frame disposed between the permeable wall and the inlet of a port of the lid, wherein the frame is configured to prevent the permeable wall from collapsing under vacuum. In some embodiments, the alternate separation member may comprise a quick connect mechanism for quick attachment and detachment of the alternate separation member.

In some embodiments, the vacuum canister may be incorporated into a system for aspirating thrombus, the system comprising an aspiration catheter comprising an elongate shaft configured for placement within a blood vessel of a subject, a supply lumen and an aspiration lumen each extending along the shaft, and an opening at or near a distal end of the supply lumen, the opening configured to allow injection of pressurized fluid into the aspiration lumen at or near the distal end of the aspiration lumen when the pressurized fluid is caused or allowed to flow through the supply lumen, a pressurized fluid source in fluid communication with the supply lumen to provide pressurized fluid to the supply lumen, and a vacuum source in fluid communication with the aspiration lumen through a vacuum canister, the vacuum canister being disposed between the aspiration catheter and the vacuum source and configured to separate thrombus from aspirant received by the aspiration catheter.

In some embodiments, the system for aspirating thrombus may comprise an aspiration catheter comprising an elongate shaft configured for placement within a blood vessel of a subject, a supply lumen and an aspiration lumen each extending along the shaft, and an opening at or near a distal end of the supply lumen, the opening configured to allow injection of pressurized fluid into the aspiration lumen at or near the distal end of the aspiration lumen when the pressurized fluid is caused or allowed to flow through the supply lumen, a tubing set comprising a first conduit having a distal end configured to couple to the aspiration lumen of the aspiration catheter and a proximal end configured to couple to a vacuum source, and a second conduit having a distal end configured to couple to the supply lumen of the aspiration catheter and a proximal end configured to couple to a first fluid source, a pressurization element configured to couple to the tubing set and further configured to pressurize fluid from the first fluid source or allow pressurized fluid from the first fluid source to be transferred to the supply lumen, such that the pressurized fluid is capable of flowing through the supply lumen from the proximal end of the supply lumen to the distal end of the supply lumen, and wherein the vacuum source comprises a vacuum canister having one or more ports, including a first port configured to couple to the first conduit of the tubing set, and wherein the vacuum canister comprises a separation member dividing the vacuum canister into an upper portion and a lower portion and an alternate separation member disposed over the first port and extending into the vacuum canister for separating thrombus removed from the subject from fluids delivered via the first conduit of the tubing set.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 is a plan view of exemplary disposable components of a system for aspirating thrombus according to an embodiment of the present disclosure.

FIG. 2 is a sectional view of an exemplary distal end of the aspiration catheter of the system for aspirating thrombus of FIG. 1.

FIG. 3 is a detail view of an exemplary y-connector of the aspiration catheter of the system for aspirating thrombus of FIG. 1.

FIG. 4 is a perspective view of exemplary disposable components of a system for aspirating thrombus according to an embodiment of the present disclosure.

FIG. 5 is an exploded perspective view of an exemplary system for aspirating thrombus of FIG. 4.

FIG. 6 is a perspective view of another aspiration catheter according to an embodiment of the present disclosure.

FIG. 7 is a cross-sectional view of another aspiration catheter according to the presentation disclosure.

FIG. 8 is a sectional view of an exemplary distal end of the aspiration catheter of FIGS. 6 and 7 aspirating thrombus according to the present disclosure.

FIG. 9 is a cross-sectional view of an exemplary vacuum canister, having a separation member disposed therein.

FIGS. 10A-10C illustrate cross-sectional views of exemplary separation members.

FIGS. 10D-10H illustrate the upper surface of exemplary separation members and apertures formed thereon.

FIGS. 11A-11B illustrate cross-sectional views of exemplary separation members.

FIG. 12 illustrates a cross-sectional view of an exemplary vacuum canister, having two separation members disposed therein.

FIG. 13 illustrates a cross-sectional view of an exemplary vacuum canister, having a moveable separation member attached to the lid of the vacuum canister.

FIG. 14 illustrates a cross-sectional view of an exemplary vacuum canister, having a moveable separation member attached to the receptacle of the vacuum canister.

FIG. 15A illustrates a cross-sectional view of an exemplary vacuum canister, having a moveable separation member attached to the lid of the vacuum canister.

FIG. 15B illustrates a top view of the moveable separation member of FIG. 15A.

FIG. 16 illustrates a cross-sectional view of an exemplary vacuum canister, having a moveable separation member and a separation member disposed therein.

FIG. 17 illustrates a cross-sectional view of an exemplary vacuum canister, having a moveable separation member and a separation member disposed therein.

FIG. 18 illustrates a detail view of an alternate separation member.

FIG. 19 is a cross-sectional view of an alternate separation member, having two permeable walls.

FIG. 20 is a cross-sectional view of an alternate separation member, having a permeable wall and a frame.

FIG. 21 is a cross-sectional view of an exemplary vacuum canister, having an alternate separation member disposed over a first port of the lid.

FIG. 22 is a cross-sectional view of an exemplary vacuum canister, having a separation member and an alternate separation member disposed therein.

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to systems, methods, and apparatus for use in aspirating thrombus, such as through using one or more fixed or movable separation members to separate liquid components of aspirated material from more viscous aspirant. The separation member can be disposed within a container in such a manner to aid with separation of liquid from more viscous aspirant. For instance, the separation member can be inclined in relation to wall(s) of a container receiving aspirated material, a moveable separation member, such as being rotatable to aid with separating more viscous aspirant from less viscous aspirant, a flaccid or flexible sack-like separation member, combinations or modifications thereof.

While the present disclosure will describe a particular implementation of various separation members, it should be understood that the devices, systems, and methods described herein may be applicable to other uses. Additionally, elements described in relation to any embodiment depicted and/or described herein may be combinable with elements described in relation to any other embodiment depicted and/or described herein.

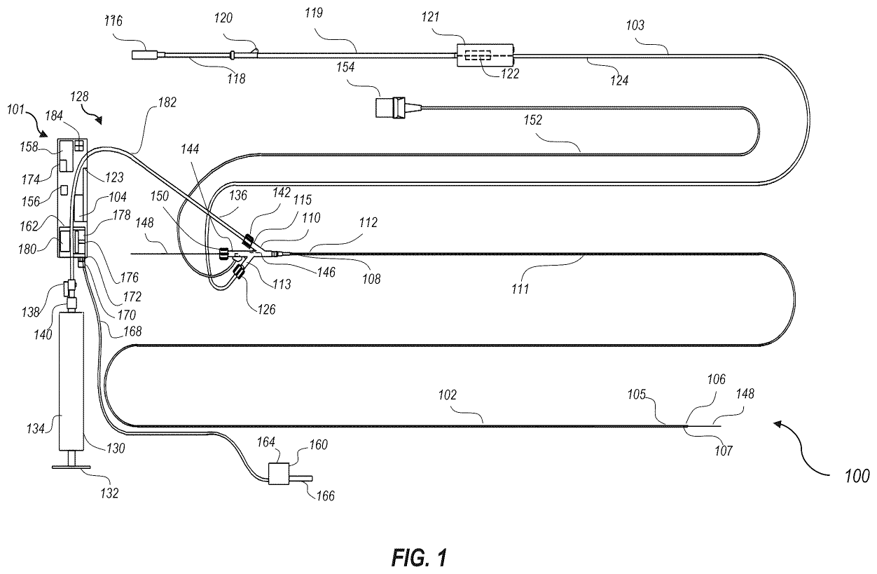

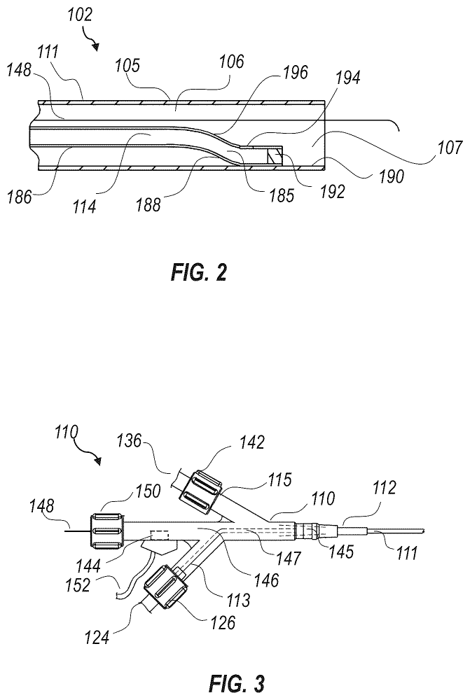

A thrombectomy system 100 for aspirating thrombus is illustrated in FIG. 1-3, illustrating primarily a distal end 105 of an aspiration catheter 102. FIGS. 2-4 illustrate the system 100 in greater detail. The system 100 for aspirating thrombus includes three major components: a pump 101, an aspiration catheter 102, and a tubing set 103. The aspiration catheter 102 and the tubing set 103 represent disposable components 104, and the pump 101, and the pump's associated pump base, is a reusable component. It is not necessary to sterilize the pump 101 as it may be kept in a non-sterile field or area during use. The aspiration catheter 102 and the tubing set 103 may each be supplied sterile, after sterilization by ethylene oxide gas, electron beam, gamma, or other sterilization methods. The aspiration catheter 102 may be packaged and supplied separately from the tubing set 103, or the aspiration catheter 102 and the tubing set 103 may be packaged together and supplied together. Alternatively, the aspiration catheter 102 and tubing set 103 may be packaged separately, but supplied together (i.e., bundled). The aspiration catheter 102 has a distal end 105 and includes an over-the-wire guidewire lumen/aspiration lumen 106 extending between an open distal end 107, and a proximal end 108 comprising a y-connector 110. While reference is made to the aspiration lumen 106 being an “over-the-wire guidewire lumen” it will be understood that the distal end 105 can include a lumen for rapid-exchange such that a guidewire need not be disposed within the aspiration lumen 106.

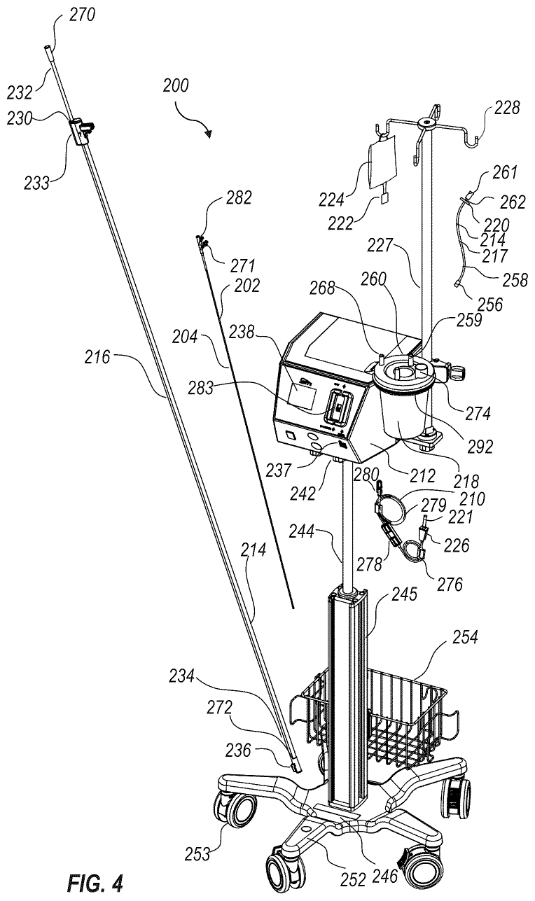

The catheter shaft 111 of the aspiration catheter 102 is connected to the y-connector 110 via a protective strain relief 112. In other embodiments, the catheter shaft 111 may be attached to the y-connector 110 with a luer fitting. The y-connector 110 comprises a first female luer 113 which communicates with a catheter supply lumen 114 (FIG. 3), and a second female luer 115 which communicates with the guidewire lumen/aspiration lumen 106.

A spike 116 for coupling to a fluid source (e.g., saline bag, saline bottle) allows fluid to enter through an extension tubing 118 and flow into a supply tube 119. An optional injection port 120 allows injection of materials or removal of air. A cassette 121 having a moveable piston 122 is used in conjunction with a mechanical actuator 123 of the pump 101. Fluid is pumped into an injection tube 124 from action of the cassette 121 as applied by the actuator 123 of the pump 101. A male luer 126, hydraulically communicating with the catheter supply lumen 114, via the injection tube 124, is configured to attach to the female luer 113 of the y-connector 110.

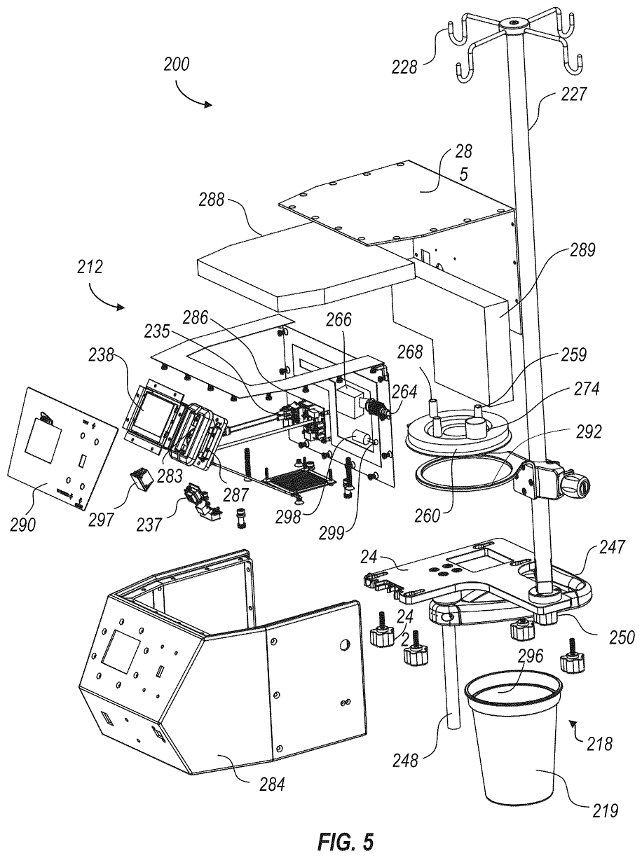

Accessories 128 are illustrated that are intended for applying a vacuum source, such as a syringe 130 having a plunger 132 and a barrel 134, to the aspiration lumen 106 of the catheter 102. The syringe 130 is attached to a vacuum line 136 via the luer 140 of the syringe 130. A stopcock 138 may be used on the luer 140 to maintain the vacuum, or alternatively, the plunger 132 may be a locking variety of plunger that is configured to be locked in the retracted (vacuum) position. A male luer 142 at the end of the vacuum line 136 may be detachably secured to the female luer 115 of the y-connector 110 of the aspiration catheter 102. As shown in more detail in FIG. 4, a pressure transducer or sensor 144 is secured inside an internal cavity 146 of the y-connector 110 proximal to the female luer 113 and the female luer 115. A valve 150, for example a Touhy-Borst, at the proximal end of the y-connector 110 allows hemostasis of the guidewire lumen/aspiration lumen 106 around a guidewire 148. In other embodiments, the valve 150 may comprise a longitudinally spring-loaded seal. The guidewire 148 may be inserted entirely through the guidewire lumen/aspiration lumen 106. Signals output from the pressure sensor 144 are carried through a cable 152 to a connector 154. The connector 154 is plugged into a socket 156 of the pump 101. Pressure related signals may be processed by a circuit board 158 of the pump 101. The pressure sensor 144 may be powered from the pump 101, via the cable 152. The accessories 128 may also be supplied sterile to the user.

A foot pedal 160 is configured to operate a pinch valve 162 for occluding or opening the vacuum line 136. The foot pedal 160 comprises a base 164 and a pedal 166 and is configured to be placed in a non-sterile area, such as on the floor, under the procedure table/bed. The user steps on the pedal 166 causing a signal to be sent along a cable 168 which is connected via a plug 170 to an input jack 172 in the pump 101. The vacuum line 136 extends through a portion of the pump 101. The circuit board 158 of the pump may include a controller 174 configured to receive one or more signals indicating on or off from the foot pedal 160. The controller 174 of the circuit board 158 may be configured to cause an actuator 176 carried by the pump 101 to move longitudinally to compress and occlude the vacuum line 136 between an actuator head 178 attached to the actuator 176 and an anvil 180, also carried by the pump 101. By stepping on the pedal 166, the user is able to thus occlude the vacuum line 136, stopping the application of a negative pressure. In some embodiments, as the pedal 166 of the foot pedal 160 is depressed, the controller may be configured to open the pinch valve 162.

The pressure sensor 144 thus senses a negative pressure and sends a signal, causing the controller to start the motor 182 of the pump 101. As the effect via the electronics is substantially immediate, the motor 182 starts pumping almost immediately after the pedal 166 is depressed. As the pedal 166 of the foot pedal 160 is released, the controller 174 then causes the pinch valve 162 to close. The pressure sensor 144 thus senses that no negative pressure is present and the controller 174 causes the motor 182 of the pump 101 to shut off. Again, the effect via the electronics is substantially immediate, and thus the motor 182 stops pumping almost immediately after the pedal 166 is depressed. During sterile procedures, the main interventionalist is usually “scrubbed” such that the hands only touch items in the sterile field. However, the feet/shoes/shoe covers are not in the sterile field. Thus again, a single user may operate a switch (via the pedal 166) while also manipulating the catheter 102 and guidewire 148. However, this time, it is the sterile field hands and non-sterile field feet that are used. Alternatively, the foot pedal 160 may comprise two pedals, one for occlude and one for open. In an alternative foot pedal embodiment, the pedal 166 may operate a pneumatic line to cause a pressure activated valve or a cuff to occlude and open the vacuum line 136, for example, by forcing the actuator head 178 to move. In another alternative embodiment, the pedal 166 may turn, slide, or otherwise move a mechanical element, such as a flexible pull cable or push rod that is coupled to the actuator 176, to move the actuator head 178. The cable 168 may be supplied sterile and connected to the base 164 prior to a procedure. The occlusion and opening of the vacuum line 136 thus acts as an on and off switch for the pump 101 (via the pressure sensor 144). The on/off function may thus be performed by a user whose hands can focus on manipulating sterile catheters, guidewires, and accessories, and whose foot can turn the pump on and off in a non-sterile environment. This allows a single user to control the entire operation or the majority of operation of the system 100 for aspirating thrombus. This can be an advantage both in terms of a rapid, synchronized procedure, but is also helpful in laboratories where additional assistants are not available. The actuator 176 and anvil 180 may be controlled to compress the vacuum line 136 with a particular force, and the actuator 176 may be controlled to move at a particular speed, either when compressing or when removing compression. Speed and force control allows appropriate response time, but may also be able to add durability to the vacuum line 136, for example, by not over compressing. The foot pedal 160 may communicate with the pinch valve 162 via a wired connection through the pump 101 or may communicate with the pinch valve 162 wirelessly. Additionally, or alternatively, the pump may be controlled by buttons 184.

It should be noted that in certain embodiments, the pinch valve 162 and the foot pedal 160 may be incorporated for on/off operation of the pinch valve 162 on the vacuum line 136, without utilizing the pressure sensor 144. In fact, in some embodiments, the pressure sensor 144 may even be absent from the system 100 for aspirating thrombus, the foot pedal 160 being used as a predominant control means.

Turning to FIG. 3, a supply tube 186, which contains the catheter supply lumen 114, freely and coaxially extends within the over-the-wire guidewire lumen/aspiration lumen 106. At least a distal end 188 of the supply tube 186 is secured to an interior wall 190 of the guidewire lumen/aspiration lumen 106 of the catheter shaft 111 by adhesive, epoxy, hot melt, thermal bonding, or other securement modalities. A plug 192 is secured within the catheter supply lumen 114 at the distal end 188 of the supply tube 186. The plug 192 blocks the exit of pressurized fluid, and thus the pressurized fluid is forced to exit through an orifice 194 in the wall 196 of the supply tube 186. The free, coaxial relationship between the supply tube 186 and the catheter shaft 111 along their respective lengths, allows for improved flexibility. In some embodiments, in which a stiffer proximal end of the aspiration catheter 102 is desired (e.g., for pushability or even torqueability), the supply tube 186 may be secured to the interior wall 190 of the guidewire lumen/aspiration lumen 106 of the catheter shaft 111 along a proximal portion of the aspiration catheter 102, but not along a distal portion. This may be appropriate if, for example, the proximal portion of the aspiration catheter 102 is not required to track through tortuous vasculature, but the distal portion is required to track through tortuous vasculature. The free, substantially unconnected, coaxial relationship between the supply tube 186 and the catheter shaft 111 along their respective lengths, may also be utilized to optimize flow through the guidewire lumen/aspiration lumen 106, as the supply tube 186 is capable of moving out of the way due to the forces of flow (e.g., of thrombus/saline) over its external surface, such that the remaining inner luminal space of the guidewire lumen/aspiration lumen 106 self-optimizes, moving toward the lowest energy condition (least fluid resistance) or toward the largest cross-sectional space condition (e.g., for accommodating and passing pieces of thrombus).

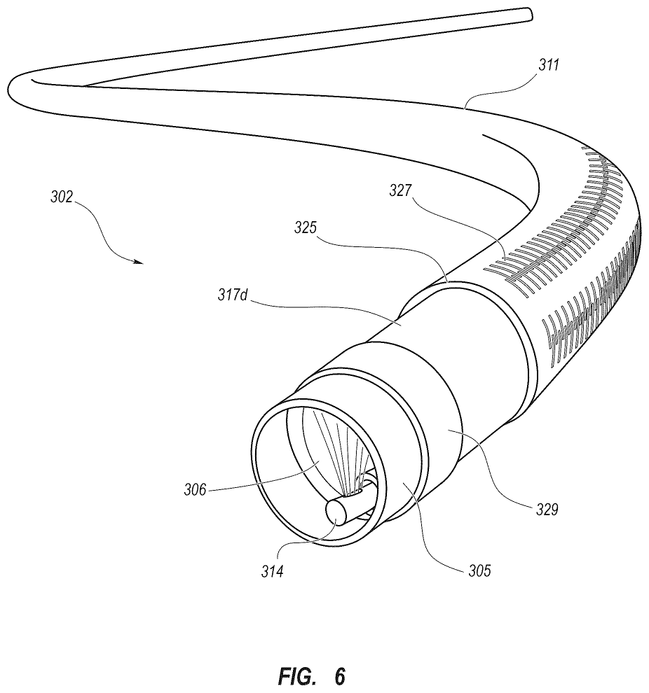

A thrombectomy system for aspirating thrombus 200 is illustrated in FIGS. 4-5. An aspiration catheter 202 is similar to the aspiration catheter 102 of FIGS. 1-3. The system 200 for aspirating thrombus is similar to the system 100 and so the disclosure related to the system 100 is also applicable to the description of system 200. An aspiration catheter 202 is similar to the aspiration catheter 102 of FIGS. 1-3 and as such the description related to the aspiration catheter 102 of FIGS. 1-3 is also applicable to the description of the aspiration catheter 202.

The aspiration catheter 202 is configured for aspirating thrombus from peripheral vessels, but may also be configured with a size for treating coronary, cerebral, pulmonary or other arteries, or veins. The aspiration catheter 202/system 200 may be used in interventional procedures, but may also be used in surgical procedures. The aspiration catheter 202/system 200 may be used in vascular procedures, or non-vascular procedures (other body lumens, ducts, or cavities). The catheter 202 comprises an elongate shaft 204 configured for placement within a blood vessel of a subject; a catheter supply lumen 114 (FIG. 3) and a guidewire/aspiration lumen 106, each extending along the shaft, the supply lumen 114 having a proximal end 147 and a distal end 185, and the aspiration lumen 106 having a proximal end 145 (FIG. 4) and an open distal end 107 (FIG. 3); and an orifice or opening 194 at or near the distal end 185 of the supply lumen 114, the opening configured to allow the injection of pressurized fluid into the aspiration lumen 106 at or near the distal end 107 of the aspiration lumen 106 when the pressurized fluid is pumped through the supply lumen 114. In some embodiments, the orifice or opening 194 may be located proximal to the distal end 185 of the supply lumen 114. In some embodiments, the distal end 185 of the supply lumen 114 may comprise a plug 192. A pump set 210 (e.g., tubing set) is configured to hydraulically couple the supply lumen 114 to a pump within a saline drive unit (SDU) 212, for injecting pressurized fluid (e.g., saline, heparinized saline) through the supply lumen 114. Suction tubing 214, comprising sterile suction tubing 216 and non-sterile suction tubing 217, is configured to hydraulically couple a vacuum canister 218 to the aspiration lumen 106. A filter 220 may be carried in-line on the suction tubing 214, for example, connected between the sterile suction tubing 216 and the non-sterile suction tubing 217, or on the non-sterile suction tubing 217. The filter 220 is configured to capture large elements such as large pieces of thrombus or emboli.

The pump set 210 includes a saline spike 221 for connection to a port 222 of a saline bag 224, and an inline drip chamber 226 for visually assessing the movement of saline, as well as keeping air out of the fluid being injected. The saline bag 224 may be hung on an IV pole 227 on one or more hooks 228. A pressure sensor 230 such as a vacuum sensor may be used within any lumen of the pump set 210, the suction tubing 214, the supply lumen 114 or aspiration lumen 106 of the catheter 202, or any other component which may see fluid flow. The pressure sensor 230 is shown in FIG. 5 within a lumen at a junction between a first aspiration tube 232 and a control 233. A cable 234 carries signals output from the pressure sensor 230 to a controller 235 in the SDU 212. A connector 236, electrically connected to the cable 234, is configured to be detachably coupled to a mating receptacle 237 (e.g., input jack) in the SDU 212. The SDU 212 also may have a display 238, including an LCD screen or alternative screen or monitor, in order to visually monitor parameters and status of a procedure. In alternative embodiments, the pressure sensor 230 may be replaced by another type of sensor that is configured to characterize fluid flow. In some embodiments, the sensor is a flow sensor, such as a Doppler flow velocity sensor.

The SDU 212 is held on a mount 240 by four locking knobs 242. The mount 240 is secured to a telescoping rod 244 that is adjustable from a cart base 245 via a cart height adjustment knob or other element 246. The mount 240 and a handle 247 are secured to the rod 244 via an inner post 248 that is insertable and securable within an inner cavity in the rod 244. The IV pole 227 secures to the mount 240 via a connector 250. The base 245 may include legs 252 having wheels 253 (e.g., three or more wheels or four or more wheels) and may be movable via the handle 247. The system 200 may also carry a basket 254 for placement of components, products, documentation, or other items.

The canister 218 may comprise a lid 260 configured to sealingly attach to a receptacle 219. In use, a user connects a first connector 256 at a first end 258 of the non-sterile suction tubing 217 to a first port 259 on the lid 260 of the canister 218, and connects a second connector 261 at a second end 262 of the non-sterile suction tubing 217 to a vacuum pump input 264 in the SDU 212. A vacuum pump 266 may be carried within the SDU 212 in order to maintain a vacuum/negative pressure within the canister 218. Alternatively, the vacuum inside the canister 218 may be maintained manually, without a vacuum pump, by evacuating the canister 218 via one or more additional ports, such as additional port 268. A user connects a first connector 270 of the sterile suction tubing 216 to an aspiration luer 271 of the aspiration catheter 202 (similar to luer 115), and connects the second connector 272 of the sterile suction tubing 216 to second port 274 on the lid 260 of the canister 218. Connector 236 is then coupled to the mating receptacle 237 in the SDU 212 for communication with the control 233 and/or the pressure sensor 230. For instance, the connector 236 can be snapped into mating receptacle 237 in the SDU 212 for communication with elements of the control 233 and/or for communication with the pressure sensor 230, either via cable 234, and/or additional cables or wires. Alternatively, the connector 236 may couple to the mating receptacle 237 by clipping, friction fitting, vacuum fitting, or other means.

After allowing saline to purge through the supply tube 276, cassette 278, and injection tube 279 of the pump set 210, the user connects the luer connector 280 of the pump set 210 to a luer 282 of the aspiration catheter 202 (similar to luer 113). The cassette 278 (similar to cassette 121) is then attached to a saddle 283 in the SDU 212. The saddle 283 is configured to reciprocate a piston to inject the saline from the IV bag 224 at high pressure, after the cassette 121 is snapped in place, keeping the internal contents (e.g., saline) sterile. Systems configured for performing this type of sterile injection of high-pressure saline are described in U.S. Pat. No. 9,883,877, issued Feb. 6, 102, and entitled, “Systems and Methods for Removal of Blood and Thrombotic Material”, which is incorporated by reference in its entirety for all purposes. The SDU 212 is enclosed within a case 284 and a case lid 285. The controller 235 may reside on a circuit board 286. Noise from a motor 287 controlling the saddle 283 and from the vacuum pump 266 is abated by internal foam sections 288, 289. The saddle 283 may be moved directly by the motor 287, or may be moved with pneumatics, using a cycled pressurization. An interface panel 290 provides one or more switches 297 and the display 238. Alternatively, the cassette 121 may couple to the saddle 283 by clipping, friction fitting, vacuum fitting, or other means.

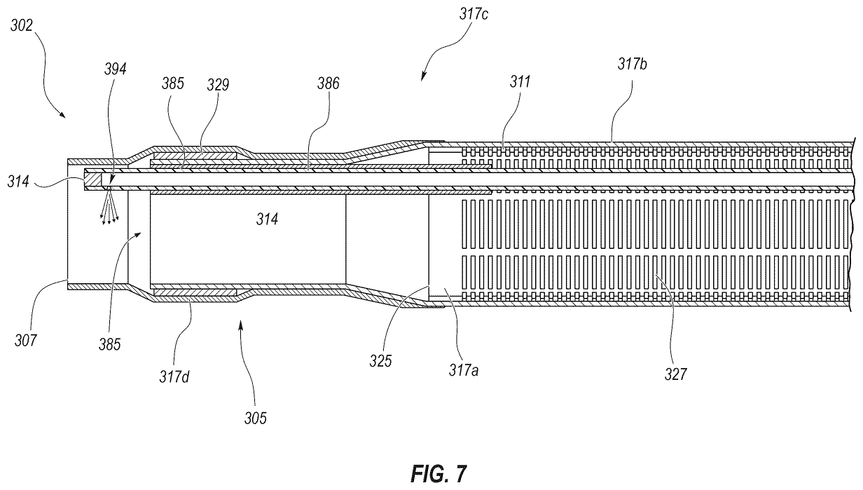

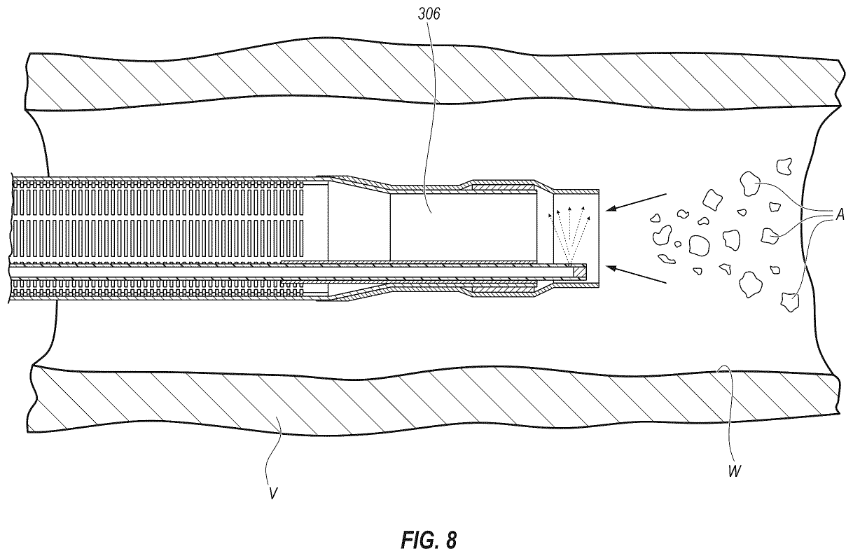

Turning to FIGS. 6-8 illustrated is another configuration of an aspiration catheter 302 that can be used with the systems 100 or 200 for aspirating thrombus. As such, the discussions related to the aspiration catheter 102 and the aspiration catheter 202 are also applicable to the aspiration catheter 302 illustrated in FIGS. 6-8.

As shown in more detail in FIGS. 6-8, aspiration catheter 302 includes an aspiration lumen 306 formed by a shaft 311, such as a hypotube, jacketed by a polymer jacket. For instance, a shaft body 317a is illustrated being jacket by a jacket 317b. A distal end 305 of the aspiration catheter 302 includes a multilayer structure. A portion 317c of the jacket 317b extends distally of a shaft distal end 325 of the shaft 311 to form part of the distal end 305. An outer jacket or layer 317d overlaps the jacket 317b, extends towards and overlaps a shaft distal end 325, and forms the aspiration catheter distal end 305 or the distal most end of the aspiration catheter. The outer jacket 317d protects a distal portion 385 of a supply tube 386 containing a supply lumen 314. While reference is made of a multilayer structure, it will be understood that one or more layers can be omitted or combined together. Additionally, one or more of the layers or shaft body can include braided or other members to increase strength and/or flexibility. Alternatively, the shaft and associated layers can be formed by extruding the shaft or using other structure to form the shaft.

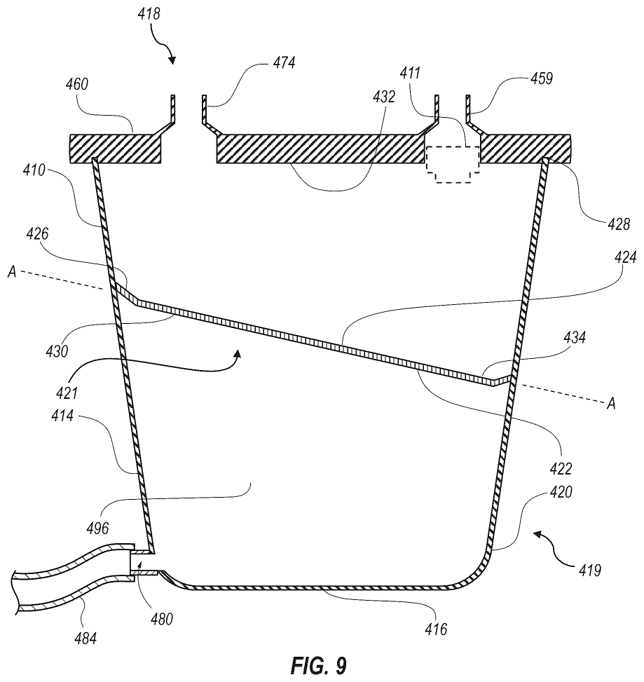

The supply lumen 314 may be configured to provide a high pressure fluid injection, such as saline, within the aspiration lumen 306 for macerating a thrombus as it is aspirated, such as illustrated in FIG. 8 where the aspiration catheter 302 is disposed within a vessel lumen W of a vessel V and aspirant A is being drawing into the aspiration lumen 306. The saline injection may occur through orifice 394 near the distal end of the supply lumen 314 if the opening of the supply lumen is plugged with a plug similar to plug 192 (FIG. 2). Aspiration catheter 302 may also include a radiopaque (RO) ring 329 at or near the distal end 305 of aspiration catheter 302 for identifying the location of aspiration. In the illustrated configuration, the RO ring 329 is disposed between the jacket 317b and outer jacket 317d. The RO ring 329 can be formed of any suitable radiopaque material, such as tantalum, tungsten, platinum/iridium, gold, silver, and combinations or modifications thereof.

The shaft 311 can include one or more openings 327 to increase a flexibility of shaft 311 to aid with advancement of the aspiration catheter 302 through the tortuous anatomy of a patient. While reference is made to a “hypotube,” it will be understood that other tubular structures can be used for the shaft 311. Additionally, the shaft 311 can be formed from polymers, metals, alloys, braided structures, coiled structures, and combinations or modifications thereof. Furthermore, the jacket 317b and outer jacket 317d can be formed of a variety of polymers and copolymers, plastics, PEBAX, HYTREL, rubber, thermoplastic elastomer, other elastomer and combinations or modifications thereof.

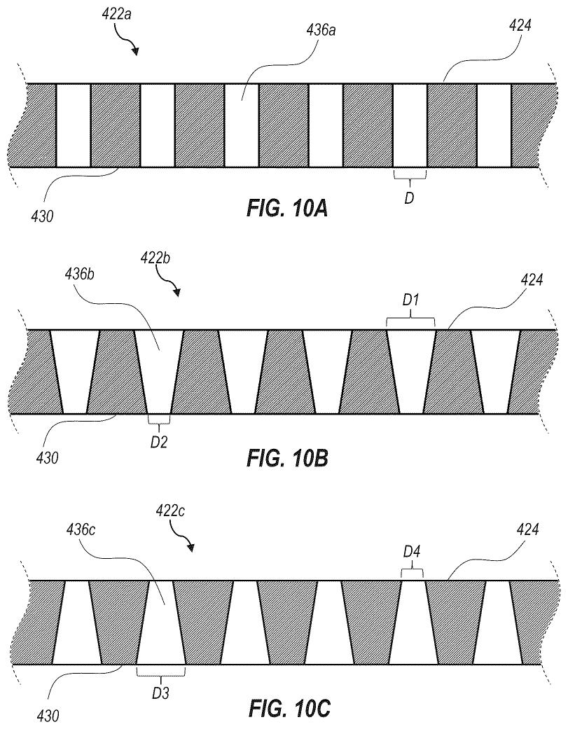

FIGS. 9-22 illustrate aspects pertaining to the vacuum canister of the aspiration system of the present disclosure. As mentioned before, thrombus or aspirant removed by the aspiration catheter is collected in the vacuum canister or canister so that a physician or clinician can inspect the same, while optionally allowing the patient and/or others to view the success of the procedure. Collection and separation of the thrombus from other liquids aids with identification and viewing of the thrombus. For instance, the canister can include one or more separation elements to aid with separating the thrombus from other liquids, such as blood, saline, etc. FIGS. 9-22 illustrate various canisters and separation structures or means for separating thrombus. As such, the disclosure of each canister/separation element or structure is also applicable to the other canister/separation element or structure and so structures and features or one canister/separation element or structure can be included within or substituted for other similar or dissimilar structures and features in other canister/separation element or structure.

Turning to FIG. 9, illustrates a cross-sectional view of the vacuum canister 418, in which aspirant (e.g., thrombus, blood, saline) that is evacuated from the patient through the aspiration lumen, such as aspiration lumens 106 and 306, is collected. The canister 418, including the lid 460, first and second ports 459, 474, and receptacle 419, and other canisters described below (including their associated lids, ports, and receptacles), may have any and all of the features of canister 218, lid 260, first and second ports 259, 274, and receptacle 219 described above. With reference to FIGS. 4, 5, and 9, the canister 418 is held in a canister mount 292 carried by the IV pole 227, or alternatively carried by any other part of the system 200, such as the SDU 212. As illustrated in FIG. 9, the vacuum canister 418 comprises a receptacle 419 and a lid 460 configured to connect to the receptacle 419 in a snapping manner to cover a portion of the receptacle 419 to close the interior 496 of the canister 418. Alternatively, the lid 460 may couple to the receptacle 419 by screwing, clipping, friction fitting, combinations or modifications thereof, or other means.

The lid 460 may comprise one or more ports, including a first port 459 for providing negative pressure/vacuum to the aspiration lumen 106. For example, the lid 460 may comprise one port, two ports, three ports, four ports, or more than four ports. Sterile suction tubing 216 may be connected to the lid 460 of the vacuum canister 418 at a second port 474 for transmitting a negative pressure to the sterile suction tubing 216 and to the aspiration lumen 106 of the aspiration catheter 102. Non-sterile suction tubing 217 may be connected to the lid 460 of the vacuum canister 418 at a first port 459 for providing a negative pressure to the vacuum canister 418. A negative pressure may be provided to the non-sterile suction tubing 217 (and to sterile suction tubing 216 and the aspiration lumen 106 connected therewith) by a vacuum source (e.g., a vacuum pump 266 or syringe). The system 200 may also comprise means for sealing the two or more ports of the lid 460 when not in use, such as one or more port caps. A filter 411 may be placed over an entry to the first port 459 so as to prevent aspirant from traveling along the non-sterile suction tubing 217 from the vacuum canister 418 to the vacuum source (e.g., the vacuum pump 266 of the SDU 212).

The receptacle 419 of the vacuum canister 418 preferably has a sufficient volumetric capacity for receiving all aspirant collected during the surgical procedure. Receptacles having a volumetric capacity of approximately 100 cubic inches, or receptacles having a diameter of approximately 5.0 inches and a height of approximately 7.0 inches, have been found to provide sufficient volumetric capacity. The walls 414 of the receptacle 419 may be relatively thin (e.g., approximately 0.10 inches to approximately 0.50 inches). Additionally, the walls 414 of the receptacle 419 may taper towards the bottom 416 of the receptacle 419, such that the diameter of an upper portion 410 of the receptacle 419 is greater than the diameter of a lower portion 420 of the receptacle 419. Alternatively, a volumetric capacity of the receptacle 419 can range from about 25 cubic inches to about 500 cubic inches, from about 50 cubic inches to about 400 cubic inches, from about 75 cubic inches to about 300 cubic inches, or from about 100 cubic inches to about 200 cubic inches, or within a range having any two of the foregoing values as endpoints. Alternatively, a diameter of the receptacle 419 can range from about 2.0 inches to about 12.0 inches, from about 4.0 inches to about 10.0 inches, or from about 6.0 inches to about 8.0 inches, or within a range having any two of the foregoing values as endpoints. Alternatively, a height of the receptacle 419 can range from about 3.0 inches to about 18.0 inches, from about 6.0 inches to about 15.0 inches, or from about 9.0 inches to about 12.0 inches, or within a range having any two of the foregoing values as endpoints.

The vacuum canister 418 may be configured to separate thrombus from other aspirant constituents (e.g., blood, saline, etc.). Such configurations of the vacuum canister 418 aid in the collection of thrombus for later analysis and enable visual confirmation by medical practitioners and patients of successful removal of thrombus. Visual confirmation may be particularly important in aiding medical practitioners to demonstrate to patients the success of the procedure and leading to increased patient satisfaction. In one embodiment, the vacuum canister 418 comprises a separation assembly 421 that includes one or more separation members 422 dividing the receptacle 419 into an upper portion 410 above the one or more separation members 422 and a lower portion 420 below the one or more separation members 422, and wherein thrombus may be collected upon an upper surface 424 of the one or more separation members 422, with saline, blood, and/or other aspirant collected in the lower portion 420 of the receptacle 419. The one or more separation members 422 may also beneficially separate the thrombus individually, without inducing thrombus clumping between individual pieces of thrombus, thereby collection of separate portions of thrombus and facilitating visualization and analysis of the thrombus thereafter. The one or more separation members 422 may be disposed at a sufficient height within the receptacle 419 such that the lower portion 420 of the receptacle has sufficient volume to collect all aspirant received during the procedure (e.g., approximately 350 ml of blood and saline) and so that aspirant settling at the bottom of the receptacle 419 does not rise to the level of the one or more separation members 422. The edge portion 426 of the one or more separation members 422 may substantially conform to the shape of the receptacle wall 414, such that the edge portion 426 of the separation member 422 follows along the receptacle wall 414. For example, the receptacle 419 having a generally cylindrical or cone-shaped (tapering) wall 414 may comprise a separation member 422 having a generally disc-like shape such that a majority or substantially all of the edge portion 426 of the separation member 422 comes in contact with the receptacle wall 414. The one or more separation members 422 may be substantially flat or may have a curved surface, such as a surface that curves toward the bottom 416 of the receptacle 419. For example, an upper surface 424 of the separation member 422 may curve towards the bottom 416 of the receptacle 419. A separation member 422 with such a curved surface may facilitate the collection of thrombus at a lowest point on the upper surface 424 of the separation member 422 and promote the visual confirmation and later sampling of the thrombus. In some embodiments, the lower surface 430 of the separation member 422 may curve toward the bottom 416 of the receptacle 419. In some embodiments, both the upper surface 424 and the lower surface 430 of the separation member 422 may curve toward the bottom 416 of the receptacle 419. Alternatively, or additionally, the edge portion 426 of the one or more separation members 422 may be bent upward, as seen in FIGS. 9, 12, 15A, 16, 17, and 22, to promote collection of thrombus towards the center of the separation member 422. The edge portion 426 of the separation member 422 may extend completely along the circumference of the upper surface 424 of the separation member 422. The edge portion 426 may be formed from the same material or a different material from the separation member 422. The edge portion 426 may comprise an elastic material, such that the edge portion 426 may be elastically deformable and capable of bending, the edge portion 426 being configured to frictionally engage the receptacle wall and prevent the separation member 422 from moving. In other embodiments, the edge portion 426 may comprise a preformed, rigid material, the edge portion 426 being configured to position the separation member 422 within the canister 418. For example, the edge portion 426 may be configured to sit atop a ledge (not shown) extending inwardly from the receptacle wall to prevent the separation member 422 from moving towards the bottom 416 of the receptacle 419. Alternatively, the edge portion 426 may be configured to slide into slots (not shown) extending into the interior surface of the receptacle wall 414 to position the separation member 422.

The one or more separation members 422 and receptacle 419 may form one composite solid structure, such that the one or more separation members 422 and receptacle 419 are formed from one material. For instance, the separation members and receptacle can be a monolithic structure.

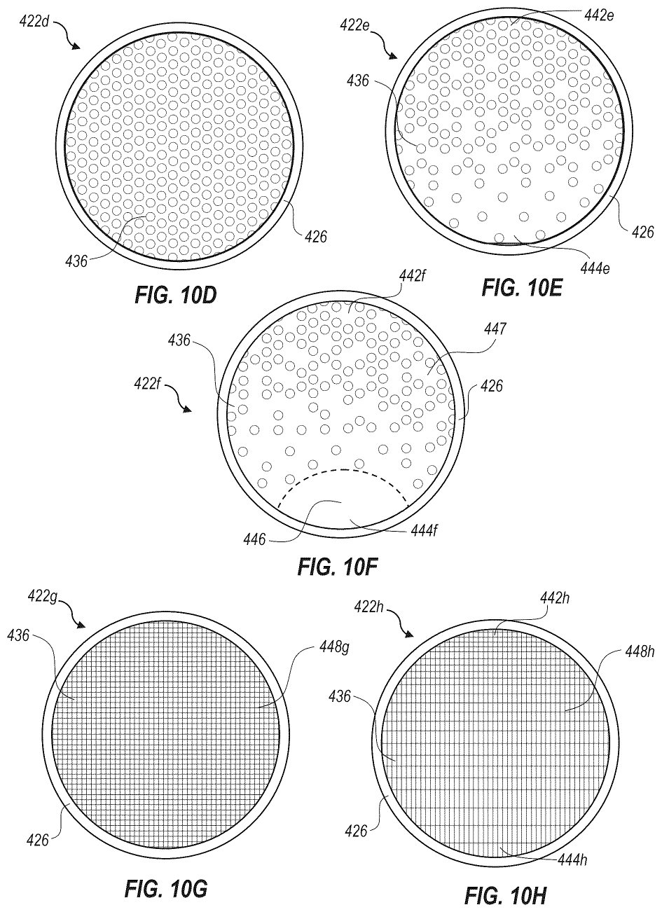

In other embodiments, the one or more separation members 422 and receptacle 419 may be formed separately. In such cases, the one or more separation members 422 may be fixed to the receptacle 419 (such as through an adhesive) or may be detachably connected to the receptacle 419 such that the one or more separation members 422 may be removed and the collected thrombus more easily sampled or that the separation member 422 may be replaced in the case of clogging of the apertures 436 (see FIGS. 10A-10H) by aspirant. For example, in embodiments wherein the edge portion 426 of the separation member 422 comprises an elastic material, a user may push the separation member 422 into the interior of the receptacle 419 such that the edge portion 426 along the circumference of the separation member 422 contacts the receptacle wall 414 and is deflected upwards. Contact between the edge portion 426 and the receptacle wall 414 may provide sufficient frictional engagement to prevent the separation member 422 from being removed while the canister 418 is under vacuum, but may be sufficiently minimal such that a user may pull upward on the separation member 422 to remove the separation member 422 upon completion of the procedure. In other embodiments, the separation member 422 may slide into slots (not shown) formed in the interior of the receptacle wall 414. For example, the separation member 422 may be placed within the receptacle 419 and attached to the canister 418 by twisting the separation member in a first direction and thereby sliding at least a section of the edge portion 426 into the slots. The separation member 422 may then be twisted in a second direction opposite that of the first direction to remove the edge portion 426 from the slots and detach the separation member 422. The one or more separation members 422 may have a relatively small thickness (e.g., approximately 0.10 inches). The one or more separation members 422 may be disposed approximately midway from the top 428 of the receptacle 419 to the bottom 416 of the receptacle 419. For example, the separation member 422 may be disposed approximately 3.0 inches to approximately 3.5 inches below the lid 460 of the vacuum canister 418 in a receptacle 419 having a height of approximately 7 inches. In embodiments wherein the separation member 422 is substantially flat, a major plane A of the separation member 422 may be oriented substantially parallel to a bottom surface 432 of the lid 460 or may be disposed at an oblique angle relative to the bottom surface 432 of the lid 460 to promote collection of thrombus to a lower side 434 of the separation member 422 (to facilitate visual confirmation and later sampling of the thrombus). The oblique angle between the separation member 422 and the bottom surface 432 of the lid 460 may be within a range of greater than 0° to approximately 30°, or approximately 5° to approximately 20°, or approximately 10° to approximately 15°, or within a range having any two of the foregoing values as endpoints.

In some embodiments, the receptacle 419 may include a port 480 that is in fluid communication with the lower portion 420 of the receptacle 419. The port 480 may allow for the aspirant constituents (e.g., blood, saline, etc.) that passed through the separation member 422 and into the lower portion 420 to be removed from the receptacle 419. In the illustrated embodiment, the port 480 is positioned in the lower portion of the wall 414 adjacent to the bottom 416. In other embodiments, the port 480 may be positioned in the bottom 416. The port 480 may be an open port or channel through the wall or bottom of the canister 418. In other embodiments, the port 480 may include a valve that may opened and closed. In some embodiments, the valve may be selectively opened and closed by a user. In other embodiments, the valve may be opened and closed in response to detected conditions. For instance, when a volume of aspirant constituents (e.g., blood, saline, etc.) in the lower portion 420 is detected to be above a predetermined threshold, the valve may open to allow for the removal of the aspirant constituents (e.g., blood, saline, etc.). Similarly, when a volume of aspirant constituents (e.g., blood, saline, etc.) in the lower portion 420 is detected to be below a predetermined threshold, the valve may close to prevent further removal of the aspirant constituents (e.g., blood, saline, etc.) or air from the canister 418.

The port 480 may be connected to a tube 484. The tube 484 may convey the aspirant constituents (e.g., blood, saline, etc.) away from the canister 418. In some embodiments, the tube 484 is connected to a pump that draws the aspirant constituents (e.g., blood, saline, etc.) out of the canister 418 and conveys them to another location. In some embodiments, the tube 484 conveys the aspirant constituents to another canister or receptacle for further analysis. In other embodiments, the tube 484 conveys the aspirant constituents to a disposal site. In yet other embodiments, the tube 484 may convey the aspirant constituents back to the patient. Conveying the aspirant constituents back to the patient may reintroduce the patient's blood that was withdrawn during the aspiration procedure. As a result, the patient may lose less blood as a result of the procedure, yet the thrombus is removed and not returned to the patient because it is captured by the separation assembly 421.

FIGS. 10A-10C illustrate a cross section of the separation member 422. The separation member 422a may include a plurality of apertures 436a extending from an upper surface 424 of the separation member 422a to a lower surface 430 of the separation member 422a. The apertures 436a may have a cross-dimension sufficiently small to allow blood and saline of the aspirate to pass therethrough, but large enough to prevent the passage of thrombotic material. The cross-dimension of the apertures 436a is measured as the greatest distance across the aperture 436a on a surface of the separation member (i.e., upper surface 424 or lower surface 430), from a first point at an edge of the aperture 436a to a second point at an edge of the aperture 436a opposite the first point, An aperture cross-dimension D of approximately 0.045 inches has been found to perform well in separating thrombus from aspirate. However, the cross-dimension D of the apertures 436 can range from about 0.010 inches to about 0.025 inches, from about 0.015 inches to about 0.125 inches, or from about 0.03 inches to about 0.0625 inches, or within a range having any two of the foregoing values as endpoints.

The apertures 436a may have a constant cross-dimension D from the upper surface 424 to the lower surface 430, as seen in FIG. 10A. FIG. 10B illustrates a separation member 422b wherein the cross-dimension of the apertures 436b may also taper from a wide cross-dimension D1 at the upper surface 424 to a narrow cross-dimension D2 at the lower surface 430. The cross-dimension of the apertures 436c may also taper from a wide cross-dimension D3 at the lower surface 430 to a narrow cross-dimension D4 at the upper surface, as seen in separation member 422c of FIG. 10C. The above tapering apertures 436b, 436c may promote the mitigation of clogging of the apertures 436b, 436c while maintaining a sufficiently small aperture cross-dimension to prevent the passage of thrombotic material. The wide cross-dimensions D1 and D3 and the narrow cross-dimensions D2 and D4 may generally fall within the ranges of cross-dimension D described above. The cross-dimension and/or tapering of the apertures 436a, 436b, 436c may be uniform for all apertures 436 of the separation member 422 or may vary from one point of the separation member 422 to another. For example, the plurality of apertures 436 may have a constant cross-dimension at a first portion of the separation member 422 and transition to a plurality of apertures 436 having tapering cross-dimension at a second portion of the separation member 422. The plurality of apertures 436 may also transition from apertures 436 having a first taper at a first portion of the separation member 422 to apertures 436 having a second taper at a second portion of the separation member 422, wherein the difference in size between the wide and narrow cross-dimensions of the tapering apertures is greater in a first portion than that at the second portion of the separation member 422.

FIGS. 10D-10H illustrate a top view of the separation member 422. The apertures 436 of the separation member 422 may be generally circular in shape, but may comprise other shapes, such as triangular, rectangular, or other polygonal shape, or may have an irregular shape. The apertures 436 may be distributed uniformly across the entirety of the upper surface 424 of the separation member 422d (as seen in FIG. 10D) or may be distributed in a non-uniform pattern upon the upper surface 424 of the separation member 422. For example, FIG. 10E illustrates that the density of the apertures 436 may decrease from a high aperture density at a first region 442e on the edge of the separation member 422e to a low aperture density at a second region 444e lying on the edge opposite the first region 442e. FIG. 10F illustrates the upper surface of a separation member 422f may include an integral portion 446 having no apertures 436 (indicated by the dotted line as a boundary between the two portion) and upon which thrombus may more easily collect, the apertures 436 being disposed upon only a portion 447 of the upper surface 424 of the separation member 422. The integral portion 446 may be disposed at or near the second region 444f opposite the high aperture density area near the first region 442f. The integral portion 446 may also be disposed at or near a lower side 434 of the separation member 422f when the separation member 422 is disposed at an angle, such that aspirate received within the canister 418 and which impinges upon the separation member 422f slides towards the integral portion 446, the blood and saline (or other liquids) of the aspirate allowed to pass through the apertures 436 with thrombus being collected upon the integral portion 446. FIG. 10G illustrates a separation member 422g comprising a mesh 448g. The mesh may be formed from intercrossing elongate members, such as wires, threads, etc. (e.g., metal wires, metal or polymeric threads, etc.), the apertures 436 formed from the space between adjacent elongate members. The cross-dimension diameter of the apertures 436 of the mesh 448g may vary depending on the space between the intercrossing elongate members. For example, the apertures 436 of the mesh 448h may increase from a first portion of the mesh 448h to a second portion of the mesh by progressively increasing the distance between parallel elongate members, as seen in FIG. 10H. A separation member 422h having such a mesh 448h will also exhibit a high aperture density region 442h that progresses to a low aperture density region 444h.

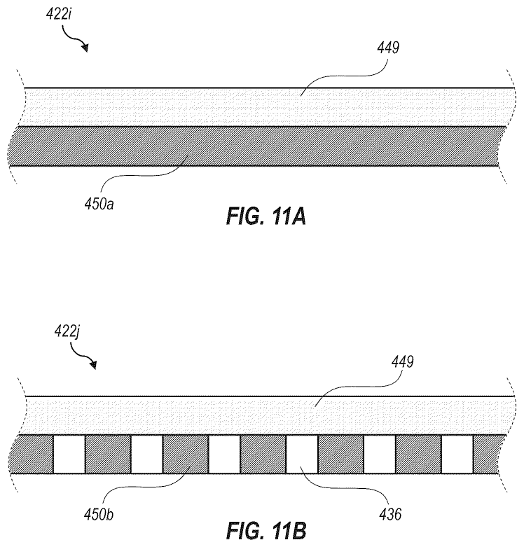

FIGS. 11A-11B illustrate a separation members 422i, 422j comprising a membrane. The separation member 422 may comprise, either wholly or in addition to embodiments described above, a membrane having a pore size sufficiently large to allow saline and blood to permeate the membrane while remaining sufficiently small to prevent further travel of thrombus. In other embodiments, the porosity of the membrane may be sufficiently small (e.g., a dialysis membrane having a pore size of approximately 5.0 nm to approximately 10.0 nm) to allow the passage of saline, but not blood, so as to separate the collected blood from saline. The membrane may comprise polymeric materials, such as polysulfone (PSU), polyethersulfone (PES), polycarbonate, polyacrylonitrile, polyamide, polyimide, polyethylene, polypropylene, polyvinylidene fluoride, and combinations or modifications thereof. The separation members 422i, 422j may comprise multiple layers. A layered separation member may act to retain the membrane within the canister. For example, as shown in FIG. 10A, the separation member 422i may include an upper layer 449 comprising a membrane and a lower layer 450a, 450b acting as a support for the upper layer. The lower layer 450b may comprise any of the features of the separation members described above, including those separation members illustrated in FIGS. 10A-10H, such as apertures 436, as shown in FIG. 11B. Generally, a pore size can range from about 0.01 μm to about 1000 μm, from about 0.1 μm inches to about 100 μm, or from about 1 μm to about 10 μm, or within a range having any two of the foregoing values as endpoints.

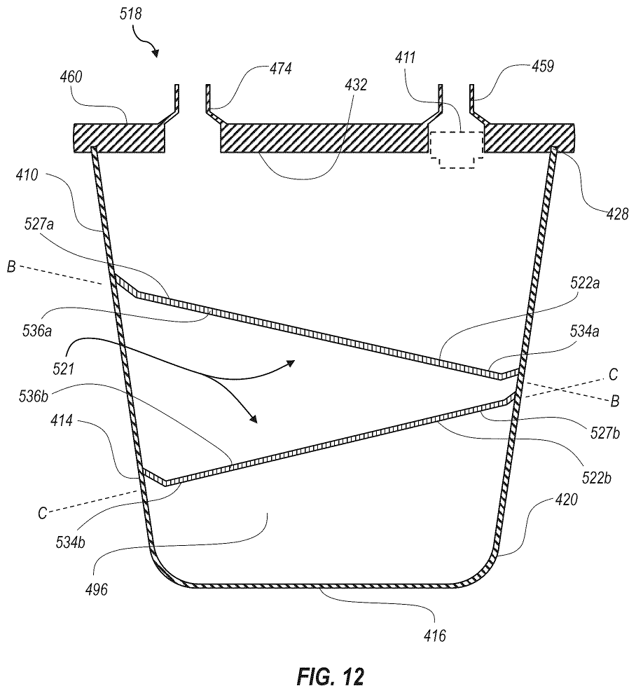

FIG. 12 illustrates a canister 518 comprising a separation assembly 521 having two separation members, including a first separation member 522a and a second separation member 522b, the first separation member 522a being disposed within the canister 518 above the second separation member 522b. The major plane B of the first separation member 522a may be oriented at an oblique angle relative to a major plane C of the second separation member 522b, such that aspirant is encouraged to migrate from a upper side 527a to a lower side 534a of the first separation member 522a and then from a upper side 527b to a lower side 534b of the second separation member 522b, so as increase the interaction between the aspirate and the collection surface of the separation members 422 and thereby promoting separation of thrombus from aspirate. The apertures 536a, 536b of the first 522a and second 522b separation members may differ in pattern, shape, cross-dimension size, and taper in a manner described above. In some embodiments, the apertures 536b are sized and configured to filter debris of between about 25 microns to about 260 microns or any value or range of values therebetween. The canister 518 may also include three separation members, four separation members, five separation members, or more than five separation members.

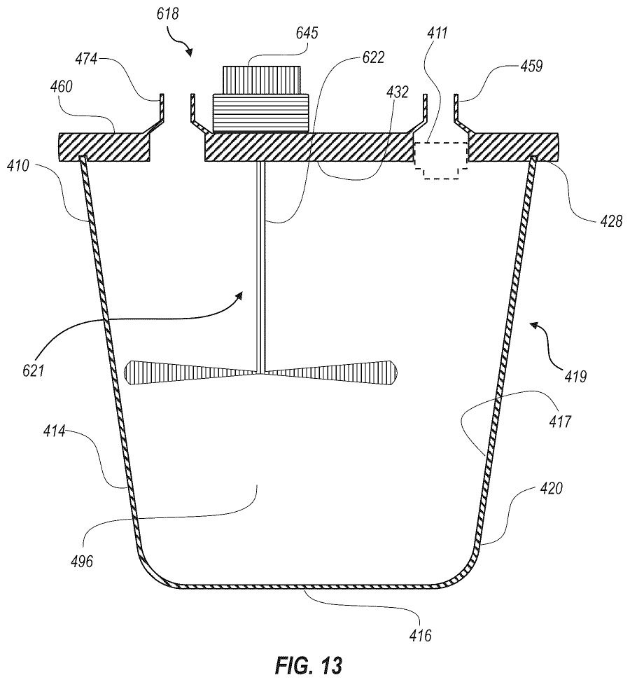

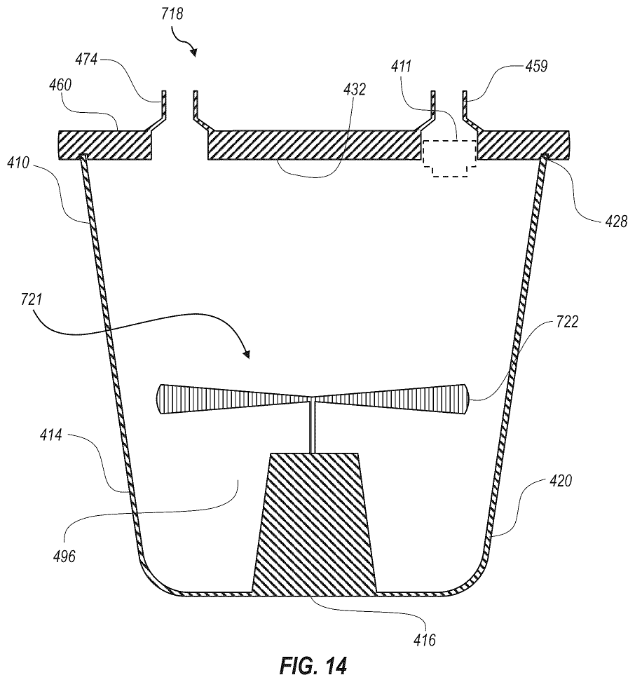

FIG. 13 illustrates a vacuum canister 618 having a separation assembly 621 comprising a moveable separation member 622, such as a rotating filter, tray, impeller, fan, or ladle, or combinations or modifications thereof. The moveable separation member 622 may be attached to the lid 460 and extend therefrom into the interior of the canister 618. FIG. 14 illustrates a separation assembly 721 wherein a moveable separation member 722 may attach to the bottom 416 of the receptacle 419 and extend upwards therefrom. The moveable separation member 622 may also be removable from the receptacle 419 or lid 460 of the canister 618. A motor 645 may be attached to the moveable separation member 622 and lid 460 to provide torque, allowing the moveable separation member 622 to rotate within the canister 618 and distribute aspirant across the walls 414 of the receptacle 419. The inner surface 417 of the receptacle walls 414 may be covered in a hydrophobic coating, such that blood and saline from aspirant will tend to slide down the wall 414, with thrombus traveling towards the bottom 416 of the canister 618 at a slower rate or adhering to the receptacle wall 414, and wherein thrombus is collected by the canister 618 on the receptacle wall 414. Collection of thrombus at the receptacle walls 414 may facilitate later removal of thrombus by an operator after the procedure (e.g., by removing the thrombus collected at the wall with a spatula), and particularly may allow individual pieces of thrombus dispersed and separated by the moveable separation member 622, 722 to be collected individually and analyzed separately. The moveable separation member 622 may be disposed beneath the second port 474 so as to ensure that all or substantially all aspirant received into the canister 618 contacts the moveable separation member 622. The canister may further be configured to reduce foaming of aspirant within the canister. Foaming of aspirant may be detrimental to an accurate evaluation of the procedure parameters, and specifically may make a determination of the volume of aspirant received into the canister (and thus the amount of blood removed from the patient) difficult. To mitigate foaming, an anti-foaming agent may be applied to the receptacle walls 414. The anti-foaming agent may, for example, comprise nitric oxide, silicones (e.g., polydimethylsiloxane), stearates, or glycols. In some embodiments, the blades (or other part of the moveable separation member rotating at a distance from the axis of rotation) of the moveable separation member 622, 722 may be placed a sufficient distance below the lid to reduce foaming. For example, as seen in FIGS. 13 and 14, the blades of the moveable separation member 622, 722 may be disposed in the bottom half of the receptacle 419.

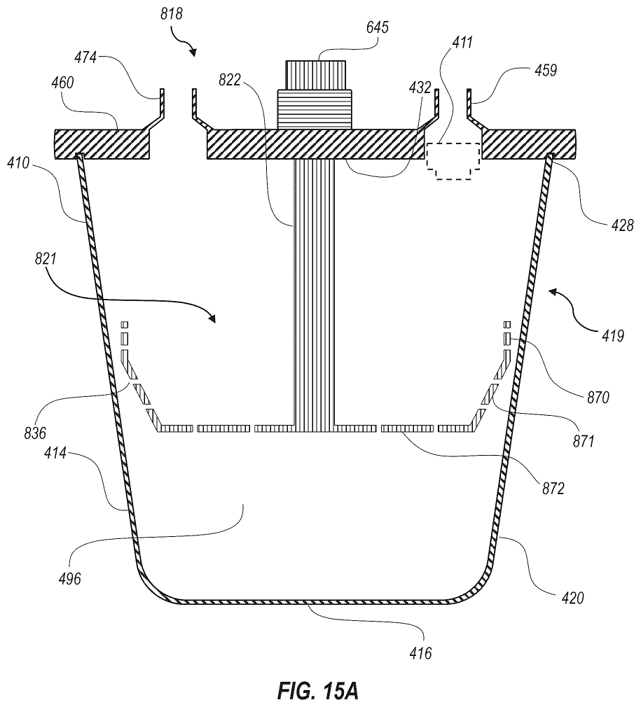

FIG. 15A illustrates a cross-sectional view of an alternative embodiment of a canister 818 having a separation assembly 821 with a moveable separation member 822 resembling a rotatable tray, in one configuration. Aspirant received into the moveable separation member 822 is flung up against the vertical 870 and slanted 871 walls as a result of the centripetal force exerted by the moveable separation member 822 on the aspirant, the slanted walls 871 enabling the separation member 822 to have a greater radius of rotation (and thus greater centripetal force) at the top of the separation member 822 than at the bottom 872 such that the separation member may generally conform in shape to the tapering walls 414 of the receptacle 419 FIG. 15B illustrates a top view of moveable separation member 822. The walls 870, 871 and bottom 872 of the moveable separation member may comprise apertures 836 similar to the apertures 836 of the separation member 422 described above, such that blood and saline, but not thrombus, may pass therethrough. Thus, thrombus may be collected in the moveable separation member 822, the aspirant being aided in passing through the apertures 836 of the moveable separation member 822 by the centripetal force imparted to the aspirant by the moveable separation member 822 when rotating. The moveable separation member 822 may have a generally circular profile, enabling the moveable separation member 822 to conform to the shape of the receptacle 419, but may comprise a profile having a square, triangular, hexagonal, shape, an irregular shape, or other shapes. The moveable separation member 822 may be detached from the lid 460 and removed after the procedure to retrieve the thrombus. The moveable separation member 822 may also be employed in combination with a separation member 422 to prevent thrombus from reaching the bottom 416 of the receptacle 419.

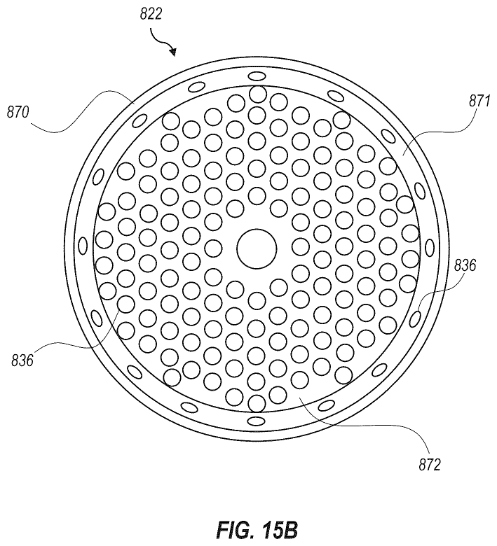

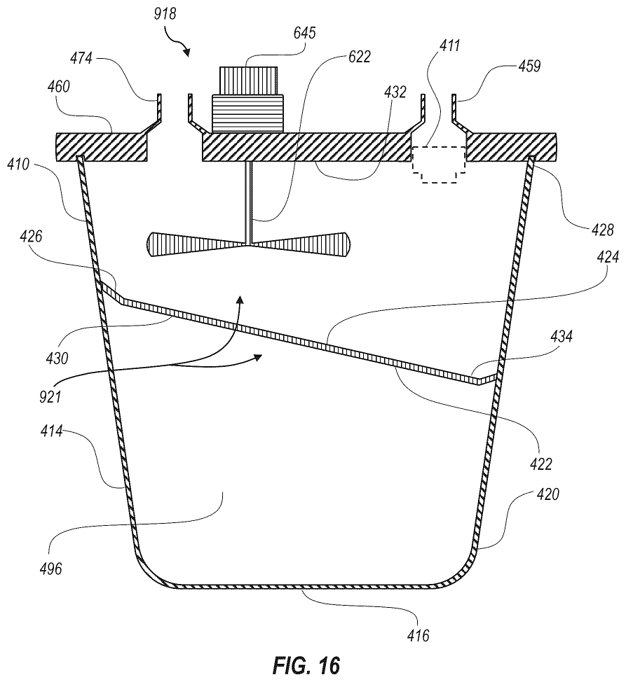

FIG. 16 illustrates a cross-sectional view of another embodiment of the canister 918 having a separation assembly 921 comprising a moveable separation member 622 and a separation member 422 similar to other separation members described above. The moveable separation member 622 acts to disperse aspirant across the surface of the separation member 422 and thereby promote the efficient passage of blood and saline across the apertures of the separation member 422. FIG. 17 illustrates a cross-sectional view of a canister 1018, wherein separation assembly 1021 includes a separation member 1022 and a moveable separation member 622, the separation member 1022 having relatively tall walls 1054 configured to receive aspirant dispersed by the moveable separation member 622. The walls 1054 of the separation member 1022 may taper at a rate similar to the tapering walls 414 of the receptacle 419 to allow the separation member 1022 to fit within the receptacle 419 and facilitate removal of the separation member 1022 from the canister after use. In some embodiments, the walls 1054 and the walls 414 may form a seal or may have a seal (e.g., O-ring) disposed therebetween to prevent non-filtered aspirant from flowing therebetween and into the lower portion 420 of the receptacle 419. The bottom 1072 of the separation member 1022 may contain apertures 1036 similar to those described above to allow blood and saline to pass therethrough.

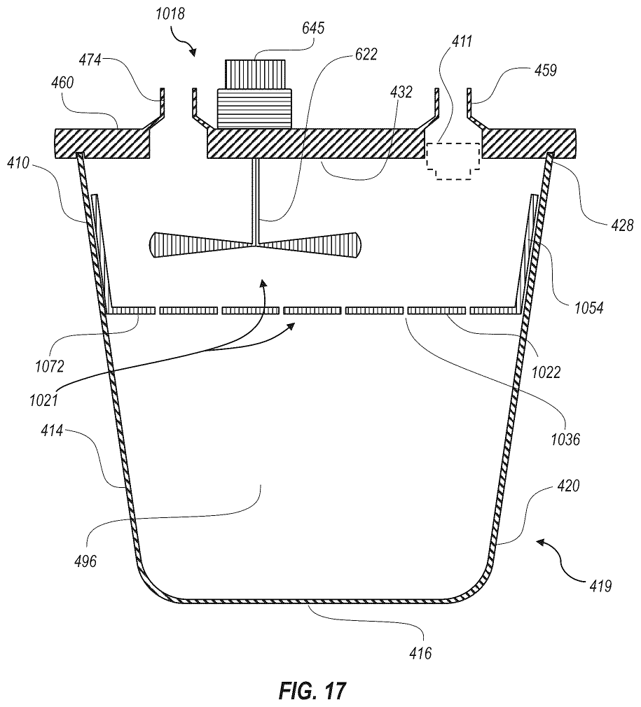

The moveable separation member 622 may have a width between 50% and 90% of the diameter of the receptacle 419, or may have a width approximately 50% of the diameter of the receptacle 419, or may have a width smaller than 50% of the diameter of the receptacle 419, or within a width range having any two of the foregoing values as endpoints. The moveable separation member 622 may be disposed below the second port 474 so as to better distribute aspirant received into the cannister 1018. FIG. 18 illustrates an exemplary alternate separation member 1122 in the form of a collection sack wherein the permeable wall 1158 of the alternate separation member 1122 comprises multiple perforations 1136 for passing blood, saline, and only small thrombotic material therethrough. The perforations 1136 of the alternate separation member 1122 may comprise various sizes and shapes and may vary in cross-dimension and shape in a manner similar to the apertures 436 of the separation member 422 described above. The perforations 1136 of the alternate separation member 1122 may also be distributed about only a portion of the permeable wall 1158 (e.g., a lower portion of the alternate separation member 1122) or may be distributed over substantially all of the permeable wall 1158. The permeable wall 1158 of the alternate separation member 1122 may also be stretchable, allowing the volume of the alternate separation member 1122 to increase as the weight of aspirant within the alternate separation member 1122 increases. Preferred materials for forming the permeable wall of the alternate separation member 1122 include silicone and latex.

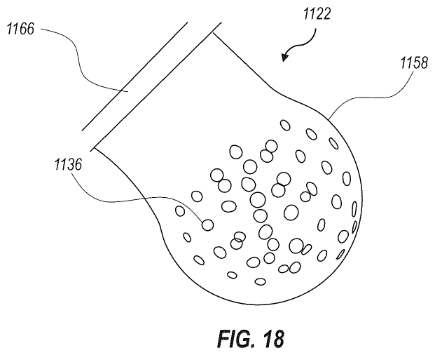

FIG. 19 illustrates a cross-sectional view of an alternate separation member 1222 having two permeable walls, including a first permeable wall 1258a disposed within a second permeable wall 1258b. The perforations 1236a and 1236b of the first 1258a and second 1258b permeable walls may vary in cross-dimension, shape, and in the proportion to which they are distributed over the respective permeable walls 1258a, 1258b. For example, the first permeable wall 1258a may include perforations 1236a having a larger cross-dimension than the perforations 1236b of the second permeable wall 1258b, or perforations 1236a may be distributed over a greater proportion of the first permeable wall 1258a than the proportion of the second permeable wall 1258b over which perforations 1236b are distributed. The alternate separation member 1222 may be detachably connected to the first port 1259 to allow for easy removal and/or replacement of the alternate separation member 1222. For example, the inlet 1261 of the alternate separation member 1222 may include a ridge 1262 that conforms to a groove 1264 formed in the first port 1259, such that alternate separation member 1222 may connect in a snapping manner to the first port 1259 by sliding the ridge 1262 of the inlet 1261 over the first port 1259 and into the groove 1264. The alternate separation member 1222 may alternatively comprise a base 1166 having a quick connect mechanism (e.g., luer lock) for quick attachment and detachment of the alternate separation member 1222, enabling the alternate separation member 1222 to be replaced in the event of clogging.

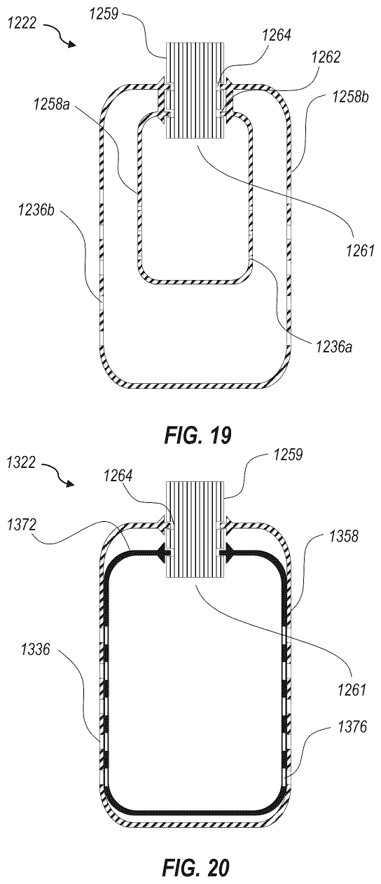

FIG. 20 illustrates a cross-sectional view of an alternate separation member 1322 comprising a permeable wall 1358 and a frame 1372. The frame 1372 may be disposed between the inlet 1261 and the permeable wall 1358, the frame providing structural support to the permeable wall 1358 to prevent the permeable wall 1358 from collapsing when under vacuum. The frame 1372 may comprise metals (e.g., aluminum, etc.) or relatively rigid plastics (e.g., polyvinyl chloride, polyethylene, polyethylene terephthalate, polypropylene, polycarbonate, etc.), and may be sufficiently rigid to withstand vacuum pressures present within the canister 818 (e.g., −1 atmosphere of vacuum). The frame may contain perforations 1376, which may comprise any of the features of the perforations of the alternate separation member described above. Perforations 1376 in the frame 1372 may substantially align with perforations 1336 of the permeable wall 1358 such that the frame 1372 does not prevent the passage of blood and saline across the permeable wall 1358.

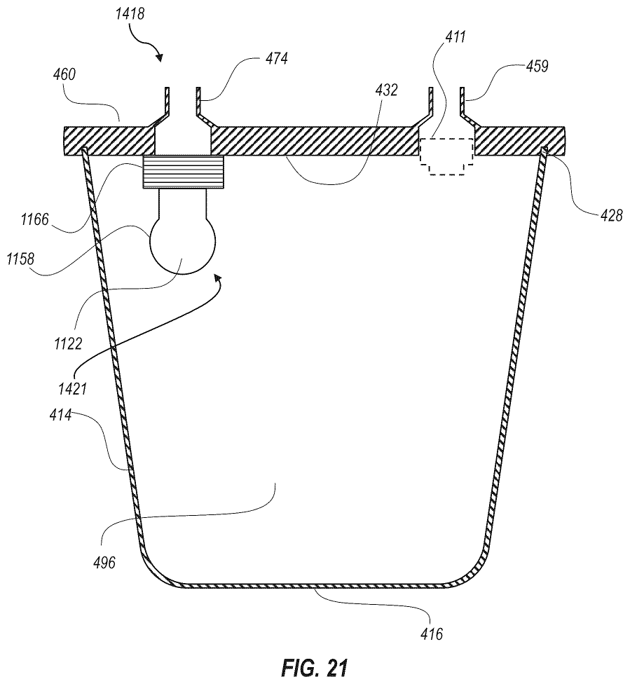

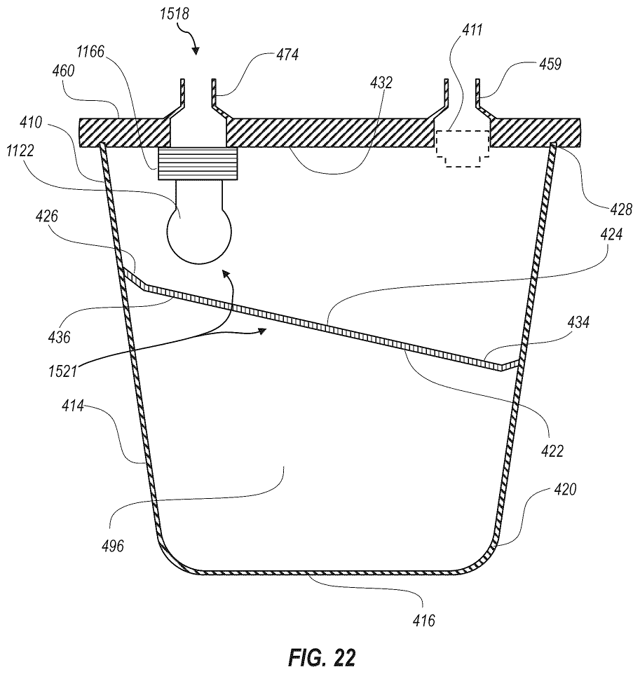

FIG. 21 illustrates a cross-sectional view of another embodiment of the vacuum canister 1418 comprising separation assembly 1421 having an alternate separation member 1122 configured to collect thrombus. The alternate separation member 1122 may include a permeable wall 1158 and may be disposed over an outlet of the second port 474 within the vacuum canister 1418, so as to separate thrombus from other constituents of aspirant. An additional benefit of the alternate separation member 1122 is that the speed of the aspirant is reduced when traveling through the alternate separation member 1122 to the interior of the receptacle 419 which thereby reduces foaming within the canister 1418. FIG. 22 illustrates a cross-sectional view of an exemplary vacuum canister 1518 having a separation assembly 1521 comprising both a separation member 422 and an alternate separation member 1122. The cross-dimension of the perforations 1136 of the alternate separation member 1122 may be larger than the cross-dimension of the apertures 436 of the separation member 422. In these embodiments, larger thrombotic material may be collected in the alternate separation member 1122, the separation member 422 further separating the aspirant, with smaller thrombotic material collected on an upper surface 424 of the separation member 422 and blood and saline collected at the bottom 416 of the receptacle 419. In some embodiments, the separation member 422 may include apertures that are sized and configured to filter debris of between about 25 microns to about 260 microns or any value or range of values therebetween. The alternate separation member 1122 may be disposed (as in FIG. 21) within the canister 1418 as the only separation member within the canister 1418, or (as in FIG. 22) may be disposed within the canister 1518 with one or more separation members 422. In other embodiments, the alternate separation member 1122 may be disposed within a canister 1418 having a moveable separation member 622, or in a canister 1418 having a moveable separation member 622 and one or more separation member 422.

The vacuum canisters 418, 518, 618, 718, 818, 918, 1018, 1418, 1518 including the receptacle 419, lid 460, and any separation member 422, 522a, 522b, 622, 722, 822, 1022, 1122, 1222, and 1322 disposed therein, may comprise materials sufficient to maintain integrity and position under negative pressure used during the procedure (e.g., −1 atmosphere of vacuum) or from forces imparted by aspirant received therein. The material of the vacuum canisters and/or separation members described above are preferably made of transparent or semi-transparent materials so as to allow visualization of collected thrombus by operators and/or patients without opening the vacuum canister and/or removing the separation member, enabling the collected thrombus to be more easily shown to the patient and providing ready evidence of success of the procedure.

Returning to FIG. 5, a solenoid 298 is carried internally in the SDU 212, and is configured to interface with the interior 296 of the canister 218, via the suction tubing 214, or via any additional tubing. The solenoid 298 is configured to vent the negative pressure inside the canister 218, by opening a valve 299 coupled to the solenoid (mechanically or electromagnetically) that opens the interior 296 of the canister 218 to ambient pressure. The venting allows any foaming of blood or fluid, such as any aspirated liquid, within the canister 218 to be reduced. Foaming can occur during a thrombolysis procedure due to cavitation, as air bubbles are formed. The solenoid 298 is then configured to close the valve 299, to allow negative pressure to again be built up within the interior 296 of the canister 218. The controller 235 is configured to automatically energize the solenoid 298, in order to allow for the degassing/defoaming. For example, the controller 235 may send a signal to energize the solenoid 298 based on the measurement of a targeted negative pressure and/or a targeted time of aspiration cycle. In other cases, the controller 235 can send a signal to energize the solenoid 298 every minute, every five minutes, every ten minutes, etc. Additionally, a user can operate the controller 235, and more generally the controller 174, of the system 200 through the interface panel 290 to initiate degassing/defoaming of the interior 296. The venting may also be able to remove air bubbles inside the other lumens of the catheter and tubing sets.

In some embodiments, the controller 235 can output or send a signal to energize the solenoid 298 to open the valve 299, in order to stop any aspiration, while still allowing the SDU 212 to deliver saline, medication, or saline combined with medication (e.g., thrombolytic drugs), so that the fluids can be delivered out of the open distal end 107 (instead of being aspirated through the aspiration lumen 106).

Although the systems for aspirating thrombus described herein are predominantly focused on aspiration, the systems may also, or alternatively, be configured for injecting or infusing fluids, with or without drugs, and may incorporate related features described in U.S. Pat. No. 10,716,583, issued Jul. 21, 2020, and entitled, “Systems and Methods for Removal of Blood and Thrombotic Material” and U.S. Pat. No. 10,492,805, issued Dec. 3, 2019, and entitled, “Systems and Methods for Thrombosis and Delivery of an Agent”.

It is contemplated that various combinations or sub-combinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present disclosure herein disclosed should not be limited by the particular disclosed embodiments described above. Moreover, while the present disclosure is susceptible to various modifications, and alternative forms, specific examples thereof have been shown in the drawings and are herein described in detail. It should be understood, however, that the present disclosure is not to be limited to the particular forms or methods disclosed, but to the contrary, the present disclosure is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

Following are some further example embodiments of the invention. These are presented only by way of example and are not intended to limit the scope of the invention in any way. Further, any example embodiment can be combined with one or more of the example embodiments.

Embodiment 1. A vacuum canister configured for use in a thrombectomy system, the canister comprising a receptacle configured to receive aspirant from a thrombectomy system, a lid configured to attach to the receptacle, the lid having one or more ports, one or more separation members configured to attach to an interior wall of the receptacle, and wherein the separation member is configured to separate thrombus from aspirant.

Embodiment 2. The vacuum canister of embodiment 1, wherein the vacuum canister comprises two or more separation members.

Embodiment 3. The vacuum canister of embodiment 1 or 2, wherein the vacuum canister comprises three or more separation members.

Embodiment 4. The vacuum canister of any one of embodiments 1 through 3, wherein at least one separation member of the one or more separation members comprises a plurality of apertures.

Embodiment 5. The vacuum canister of embodiment 4, wherein at least a portion of the plurality of apertures extend from a top surface of the at least one separation member to a bottom surface of the at least one separation member.

Embodiment 6. The vacuum canister of embodiment 4 or 5, wherein a first density of apertures of a first portion of the plurality of apertures varies from a second density of apertures of a second portion of the plurality of apertures.

Embodiment 7. The vacuum canister of any one of embodiments 4 to 6, wherein a cross-dimension of the apertures within at least a portion of the plurality of apertures tapers from a wide aperture cross-dimension to a narrow aperture cross-dimension.

Embodiment 8. The vacuum canister of any one of embodiments 4 to 7, wherein a first aperture cross-dimension of a first portion of the plurality of apertures varies from a second aperture cross-dimension of a second portion of the plurality of apertures.

Embodiment 9. The vacuum canister of any one of embodiments 4 to 8, wherein at least one aperture of the plurality of apertures has a cross-dimension of 0.045 inches.

Embodiment 10. The vacuum canister of any one of embodiments 4 to 9, wherein the at least one separation member is disposed at an oblique angle relative to a top surface of the vacuum canister.

Embodiment 11. The vacuum canister of embodiment 10, wherein the oblique angle of the at least one separation member is within a range of greater than 0° to approximately 30°.

Embodiment 12. The vacuum canister of any one of embodiments 4 to 11, wherein the at least one separation member comprises a membrane.

Embodiment 13. The vacuum canister of any one of embodiments 1 through 12, wherein at least a portion of the vacuum canister is configured to be transparent or semi-transparent for viewing collected thrombus.

Embodiment 14. A vacuum canister configured for use in a thrombectomy system, the canister comprising a receptacle configured to receive aspirant from a thrombectomy system, a lid configured to attach to the receptacle to form an interior space, the lid having one or more ports, a moveable separation member, at least a portion of which is disposed within the interior space, and wherein the moveable separation member is configured to deliver aspirant received into the vacuum canister onto an interior wall of the receptacle.

Embodiment 15. The vacuum canister of embodiment 14, wherein the moveable separation member is disposed beneath an outlet of a first port through which aspirant is received into the vacuum canister.

Embodiment 16. The vacuum canister of embodiment 14 or 15, wherein the moveable separation member is connected to the lid of the vacuum canister.

Embodiment 17. The vacuum canister of embodiment 14 or 15, wherein the moveable separation member is connected to the receptacle of the vacuum canister.

Embodiment 18. The vacuum canister of any one of embodiments 14 to 17, wherein at least a portion of the vacuum canister is configured to be transparent or semi-transparent for viewing collected thrombus.

Embodiment 19. A vacuum canister configured for use in a thrombectomy system, the canister comprising a receptacle configured to receive aspirant from a thrombectomy system, a lid configured to attach to the receptacle to form an interior space and wherein the lid includes a port, an alternate separation member, at least a portion of which is disposed within the interior space, wherein the alternate separation member is configured to connect to the lid, wherein the alternate separation member is disposed over an outlet of the port, and wherein the alternate separation member is configured to separate thrombus from aspirant received through the port.

Embodiment 20. The vacuum canister of embodiment 19, wherein the alternate separation member is configured to detachably connect to the lid.

Embodiment 21. The vacuum canister of embodiment 19 or 20, wherein the alternate separation member further comprises a first permeable wall.

Embodiment 22. The vacuum canister of embodiment 21, wherein the alternate separation member further comprises a second permeable wall configured to surround at least a portion of the first permeable wall.

Embodiment 23. The vacuum canister of embodiment 22, wherein the first and second permeable walls comprise a plurality of perforations.

Embodiment 24. The vacuum canister of embodiment 23, wherein at least a first portion of the plurality of perforations disposed upon the first permeable wall have a cross-dimension larger than a cross-dimension of a second portion of the plurality of perforations disposed upon the second permeable wall.

Embodiment 25. The vacuum canister of any one of embodiments 19 to 24, wherein the alternate separation member comprises silicone or latex.

Embodiment 26. The vacuum canister of any one of embodiments 19 to 25, wherein at least a portion of the vacuum canister is configured to be transparent or semi-transparent for viewing collected thrombus.

Embodiment 27. A system for aspirating thrombus, comprising an aspiration catheter comprising an elongate shaft configured for placement within a blood vessel of a subject, a supply lumen and an aspiration lumen each extending along the shaft, and an opening at or near a distal end of the supply lumen, the opening configured to allow injection of pressurized fluid into the aspiration lumen at or near the distal end of the aspiration lumen when the pressurized fluid is caused or allowed to flow through the supply lumen, a pressurized fluid source in fluid communication with the supply lumen to provide pressurized fluid to the supply lumen, and a vacuum source in fluid communication with the aspiration lumen through a vacuum canister, the vacuum canister being disposed between the aspiration catheter and the vacuum source and configured to separate thrombus from aspirant received by the aspiration catheter.

Embodiment 28. The system of embodiment 27, wherein the vacuum canister comprises one or more separation members configured to attach to an interior wall of the vacuum canister.

Embodiment 29. The system of embodiment 28, wherein the vacuum canister comprises two or more separation members.

Embodiment 30. The system of embodiment 28 or 29, wherein the vacuum canister comprises three or more separation members.

Embodiment 31. The system of any one of embodiments 28 to 30, wherein at least one separation member of the one or more separation members comprises a plurality of apertures.

Embodiment 32. The system of embodiment 31, wherein at least a portion of the plurality of apertures extends from a top surface of the at least one separation member to a bottom surface of the at least one separation member.

Embodiment 33. The system of embodiment 31 or 32, wherein a first density of apertures of a first portion of the plurality of apertures varies from a second density of apertures of a second portion of the plurality of apertures.

Embodiment 34. The system of any one of embodiments 31 to 33, wherein a cross-dimension of the apertures within at least a portion of the plurality of apertures tapers from a wide aperture cross-dimension to a narrow aperture cross-dimension.

Embodiment 35. The system of any one of embodiments 31 to 34, wherein a first aperture cross-dimension of a first portion of the plurality of apertures varies from a second aperture cross-dimension of a second portion of the plurality of apertures.

Embodiment 36. The system of any one of embodiments 31 to 35, wherein at least one aperture of the plurality of apertures has a cross-dimension of 0.045 inches.

Embodiment 37. The system of any one of embodiments 31 to 36, wherein the at least one separation member is disposed at an oblique angle relative to a top surface of the vacuum canister.

Embodiment 38. The system of embodiment 37, wherein the oblique angle of the at least one separation member is within a range of greater than 0° to approximately 30°.

Embodiment 39. The system of any one of embodiments 31 to 38, wherein the at least one separation member comprises a membrane.

Embodiment 40. The system of embodiment 27, wherein the vacuum canister further comprises a receptacle configured to receive aspirant from the aspiration catheter, a lid configured to attach to the receptacle to form an interior space, wherein the lid includes a port.

Embodiment 41. The system of embodiment 40, wherein the vacuum canister further comprises a moveable separation member, at least a portion of which is disposed within the interior space, and wherein the moveable separation member is configured to deliver aspirant received into the vacuum canister onto an interior wall of the receptacle.

Embodiment 42. The system of embodiment 41, wherein the moveable separation member is disposed beneath an outlet of a first port through which aspirant is received into the vacuum canister.

Embodiment 43. The system of embodiment 41 or 42, wherein the moveable separation member is connected to the lid of the vacuum canister.

Embodiment 44. The system of embodiment 41 or 42, wherein the moveable separation member is connected to the receptacle of the vacuum canister.

Embodiment 45. The system of embodiment 40, wherein the vacuum canister further comprises an alternate separation member, at least a portion of which is disposed within the interior space, wherein the alternate separation member is configured to connect to the lid, wherein the alternate separation member is disposed over an outlet of the port, and wherein the alternate separation member is configured to separate thrombus from aspirant received through the port.

Embodiment 46. The system of embodiment 45, wherein the alternate separation member is configured to detachably connect to the lid.

Embodiment 47. The system of embodiment 45 or 46, wherein the alternate separation member further comprises a first permeable wall.

Embodiment 48. The system of embodiment 47, wherein the alternate separation member further comprises a second permeable wall configured to surround at least a portion of the first permeable wall.

Embodiment 49. The system of embodiment 48, wherein the first and second permeable walls comprise a plurality of perforations.

Embodiment 50. The system of embodiment 49, wherein at least a first portion of the plurality of perforations disposed upon the first permeable wall have a cross-dimension larger than a cross-dimension of a second portion of the plurality of perforations disposed upon the second permeable wall.

Embodiment 51. The system of any one of embodiments 45 to 50, wherein the alternate separation member comprises silicone or latex.

Embodiment 52. The system of any one of embodiments 27 to 51, wherein at least a portion of the vacuum canister is configured to be transparent or semi-transparent for viewing thrombus collected in the vacuum canister.

Embodiment 53. The system of any one of embodiments 27 to 52, wherein the vacuum canister comprises a dialysis membrane configured to separate blood from saline.

Embodiment 54. The system of any one of embodiments 27 to 53, wherein the vacuum canister comprises a second port coupled to a vacuum, such that the vacuum canister is placed under vacuum via the second port.

Embodiment 55. The system of embodiment 54, wherein a filter is disposed over the second port of the vacuum canister to prevent aspirant from traveling through the second port.

Embodiment 56. The system of any one of embodiments 27 to 55, wherein the vacuum canister has a volumetric capacity of approximately 100 cubic inches.

Embodiment 57. A system for aspirating thrombus, comprising an aspiration catheter comprising an elongate shaft configured for placement within a blood vessel of a subject, a supply lumen and an aspiration lumen each extending along the shaft, and an opening at or near a distal end of the supply lumen, the opening configured to allow injection of pressurized fluid into the aspiration lumen at or near the distal end of the aspiration lumen when the pressurized fluid is caused or allowed to flow through the supply lumen, a tubing set comprising a first conduit having a distal end configured to couple to the aspiration lumen of the aspiration catheter and a proximal end configured to couple to a vacuum source, and a second conduit having a distal end configured to couple to the supply lumen of the aspiration catheter and a proximal end configured to couple to a first fluid source, a pressurization element configured to couple to the tubing set and further configured to pressurize fluid from the first fluid source or allow pressurized fluid from the first fluid source to be transferred to the supply lumen, such that the pressurized fluid is capable of flowing through the supply lumen from the proximal end of the supply lumen to the distal end of the supply lumen, and wherein the vacuum source comprises a vacuum canister having one or more ports, including a first port configured to couple to the first conduit of the tubing set, and wherein the vacuum canister comprises a separation member dividing the vacuum canister into an upper portion and a lower portion and an alternate separation member disposed over the first port and extending into the vacuum canister for separating thrombus removed from the subject from fluids delivered via the first conduit of the tubing set.

Embodiment 58. The system of embodiment 57, wherein the separation member comprises a plurality of apertures, the alternate separation member comprises a plurality of
perforations, and wherein a cross-dimension of the perforations of the plurality of perforations is greater than a cross-dimension of the apertures of the plurality of apertures.

1. A vacuum canister configured for use in a thrombectomy system, the canister comprising:
a receptacle configured to receive aspirant from a thrombectomy system,
a lid configured to attach to the receptacle, the lid having one or more ports,
a separation member configured to attach to an interior wall of the receptacle, wherein the separation member is configured to separate thrombus from aspirant.

2. The vacuum canister of claim 1, wherein the vacuum canister comprises two or more separation members or three or more separation members.

3. The vacuum canister of claim 1, the separation member comprises a plurality of apertures.

4. The vacuum canister of claim 3, wherein at least a portion of the plurality of apertures extend from a top surface of the separation member to a bottom surface of the separation member.

5. The vacuum canister of claim 3, wherein a first density of apertures of a first portion of the plurality of apertures varies from a second density of apertures of a second portion of the plurality of apertures.

6. The vacuum canister of claim 3, wherein a cross-dimension of the apertures within at least a portion of the plurality of apertures tapers from a wide aperture cross-dimension to a narrow aperture cross-dimension.

7. The vacuum canister of claim 3, wherein a first aperture cross-dimension of a first portion of the plurality of apertures varies from a second aperture cross-dimension of a second portion of the plurality of apertures.

8. The vacuum canister of claim 3, wherein at least one aperture of the plurality of apertures has a cross-dimension of 0.045 inches.

9. The vacuum canister of claim 3, wherein the separation member is disposed at an oblique angle relative to a top surface of the vacuum canister.

10. The vacuum canister of claim 9, wherein the oblique angle of the at least one separation member is within a range of greater than 0° to approximately 30°.

11. The vacuum canister of claim 3, wherein the separation member comprises a membrane.

12. The vacuum canister of claim 1, wherein at least a portion of the vacuum canister is configured to be transparent or semi-transparent for viewing collected thrombus.

13. A vacuum canister configured for use in a thrombectomy system, the canister comprising:
a receptacle configured to receive aspirant from a thrombectomy system,
a lid configured to attach to the receptacle to form an interior space, the lid having one or more ports,
a moveable separation member, at least a portion of which is disposed within the interior space, wherein the moveable separation member is configured to deliver aspirant received into the vacuum canister onto an interior wall of the receptacle.

14. The vacuum canister of claim 13, wherein the moveable separation member is disposed beneath an outlet of a first port through which aspirant is received into the vacuum canister.

15. The vacuum canister of claim 14, wherein the moveable separation member is connected to the lid or the receptacle of the vacuum canister.

16. The vacuum canister of claim 13, wherein at least a portion of the vacuum canister is configured to be transparent or semi-transparent for viewing collected thrombus.

17. A vacuum canister configured for use in a thrombectomy system, the canister comprising:
a receptacle configured to receive aspirant from a thrombectomy system,
a lid configured to attach to the receptacle to form an interior space and wherein the lid includes a port,
an alternate separation member, at least a portion of which is disposed within the interior space,
wherein the alternate separation member is configured to connect to the lid,
wherein the alternate separation member is disposed over an outlet of the port, and
wherein the alternate separation member is configured to separate thrombus from aspirant received through the port.

18. The vacuum canister of claim 17, wherein the alternate separation member further comprises a first permeable wall.

19. The vacuum canister of claim 18, wherein the alternate separation member further comprises a second permeable wall configured to surround at least a portion of the first permeable wall.

20. The vacuum canister of claim 19, wherein the first and second permeable walls comprise a plurality of perforations.

21. The vacuum canister of claim 20, wherein at least a first portion of the plurality of perforations disposed upon the first permeable wall have a cross-dimension larger than a cross-dimension of a second portion of the plurality of perforations disposed upon the second permeable wall.

22. The vacuum canister of claim 17, wherein the alternate separation member comprises silicone or latex.
